(19) United States
(12) Patent Application Publication
(10) Pub. No.: US 2022/0401574 A1
(43) Pub. Date: Dec. 22, 2022

(54) CARDIOPROTECTIVE MICROBUBBLE-LIPOSOMAL DRUG COMPLEXES

(71) Applicant: UNIVERSITY OF PITTSBURGH - OF THE COMMONWEALTH SYSTEM OF HIGHER EDUCATION, Pittsburgh, PA (US)

(72) Inventors: Flordeliza S. Villanueva, Pittsburgh, PA (US); Xucai Chen, Glenshaw, PA (US)

(21) Appl. No.: 17/773,974

(22) PCT Filed: Nov. 13, 2020
(86) PCT No.: PCT/US20/60435

§ 371 Date: May 3, 2022

Related U.S. Application Data
(60) Provisional application No. 62/935,754, filed on Nov. 15, 2019.

(51) Int. Cl. A61K 47/69; A61K 41/00; A61K 49/22; A61K 47/66; A61K 9/16; A61P 35/00

(52) U.S. Cl. CPC A61K 47/6925; A61K 41/0028; A61K 49/223; A61K 47/665; A61K 9/1658; A61P 35/00

(57) ABSTRACT

The presently disclosed drug-loaded liposomal conjugated to polymer microbubbles showed: i) increased tumor drug concentration; ii) reduced tumor growth; and ii) increased survival time in a mouse cancer model when exposed to concurrent high and low acoustic pressure ultrasonic pulses as compared to individual high or low acoustic pressure ultrasonic pulses. Notably, when unconjugated drug-loaded liposome were administered with free microbubbles and exposed to concurrent high and low acoustic pressure ultrasonic pulses, a superior tumor growth inhibition was also seen. Three weeks after treatments, DoxLPX+US group showed significantly better left ventricular function indices from echocardiography imaging than the free Dox group. Clinical methods using these liposomal conjugated microbubbles permit an increased therapeutic drug delivery and improved safety profile, respectively due to enhanced, preferential drug accumulation in target tumor tissue and simultaneously reduced drug delivery to non-target tissue.

### STATEMENT REGARDING FEDERAL FUNDING

This invention was made with government support under Grant No. EB019582 awarded by the National Institutes of Health. The government has certain rights in the invention.

### FIELD OF THE INVENTION

The present invention is directed towards improved methods of treating cancer tumors. In particular, the invention inhibits tumor growth without impairing the function of non-cancerous tissues. For example, a systemically administered and ultrasonically-directed chemotherapeutic-loaded liposome conjugated to a polymer microbubble inhibits tumor growth on a cancerous tissues without impairing the function of non-cancerous tissues.

### BACKGROUND OF THE INVENTION

Doxorubicin (Dox) is standard of care for treatment of sarcomas; tumor response correlates with dosage. Nonetheless, it is well known that Dox is cardiotoxic in a dose-dependent manner, which can limit maximum dosing for effective therapy.

Microbubbles (MBs) are intravenously injectable gas microspheres that are clinically used as ultrasound (US) contrast agents. MBs can also be used as drug or gene carriers that undergo US-triggered unloading of cargo at disease-specific sites via navigation of the US beam. It was previously found that the efficacy of liposomal-Dox-MB complexes (DoxLPX) with either high or low US pressures for inhibiting tumor cell growth in vitro was equivalent to free Dox. Yu et al., Mol Pharm 13:55-64 (2015).

What is needed in the art is a US regimen that releases a therapeutic drug from a microbubble that results in improved tumor growth suppression and minimal non-target tissue toxicity as compared to equivalent doses of the free drug.

### SUMMARY OF THE INVENTION

The present invention is directed towards improved methods of treating cancer tumors. In particular, the invention inhibits tumor growth without impairing the function of non-cancerous tissues. For example, a systemically administered and ultrasonically-directed chemotherapeutic-loaded liposome conjugated to a polymer microbubble inhibits tumor growth on a cancerous tissues without impairing the function of non-cancerous tissues.

In one embodiment, the present invention contemplates a method, comprising: a) providing; i) a patient comprising a cancerous tumor and at least one cardiac function parameter, said cancerous tumor having a volume; ii) a pharmaceutically acceptable composition comprising a doxorubicin-lipopolycomplex and a polymer microbubble; iii) an ultrasonic delivery system (e.g., a transducer) configured to generate a plurality of combined ultrasonic waves, wherein said plurality of combined ultrasonic wave comprises a first ultrasonic pulse having a higher acoustic pressure than a second ultrasonic pulse having lower acoustic pressure; b) injecting said pharmaceutically acceptable composition into said patient; c) exposing said cancerous tumor to at least one of said plurality of combined ultrasonic waves concurrent and following with said injecting; d) reducing said cancerous tumor volume without a clinical impairment of said patient in at least one cardiac function parameter. In one embodiment, the doxorubicin-lipopolycomplex is conjugated to said polymer microbubble. In one embodiment, the at least one cardiac function parameter is selected from the group consisting of ejection fraction, fractional shortening, left ventricular systolic function and left ventricular mass. In one embodiment, the clinical impairment of said at least one cardiac function parameter is greater than 30% of a baseline value. In one embodiment, the polymer microbubble comprises a polylactide polymer and nitrogen gas. In one embodiment, the polymer microbubble has an internal diameter ranging from 0.5 to 10 μm. In one embodiment, the conjugation of liposomes onto to the bubble surface is via avidin-biotin bridging. In one embodiment, the polymer microbubble is coated with an albumin. In one embodiment, the first ultrasonic pulse has an acoustic pressure selected from the group consisting of at least 500 kPa, 500 to 1500 kPa, 900 to 1100 kPa and 1000 kPa. In one embodiment, the second ultrasonic pulse has an acoustic pressure selected from the groups consisting of less than 250 kPa, 150-200 kPa, 170-180 kPa and 174 kPa. In one embodiment, the combined ultrasound wave has a frequency selected from the group consisting of 0.5 MHz to 1.0 MHZ, 0.75 MHz to 1.25 MHz, 0.9 MHz to 1.1 MHz, 1.0 MHz to 10.0 MHz, and 1.0 MHz. In one embodiment, the combined ultrasonic wave has a cycle number selected from the group consisting of 5 to 25 cycles, at least 25 cycles, 25 to 1000 cycles, 50 to 150 cycles and 95 to 105 cycles.

In one embodiment, the present invention contemplates a method, comprising: a) providing; i) a patient comprising a diseased tissue and at least one non-diseased tissue function parameter, said diseased tissue having at least one symptom; ii) a pharmaceutically acceptable composition comprising a drug-lipopolycomplex and a microbubble; iii) an ultrasonic delivery system (e.g., a transducer) configured to generate a plurality of combined ultrasonic waves, wherein said plurality of combined ultrasonic wave comprises a first ultrasonic pulse having a higher acoustic pressure than a second ultrasonic pulse having lower acoustic pressure; b) injecting said pharmaceutically acceptable composition into said patient; c) exposing said diseased tissue to at least one of said plurality of combined ultrasonic waves concurrent and following with said injecting; d) reducing said at least one symptom of said diseased tissue without a clinical impairment of said patient in said at least one non-diseased tissue function parameter. In one embodiment, the drug-lipopolycomplex is conjugated to said polymer microbubble. In one embodiment, the clinical impairment of said at least one non-diseased tissue function parameter is greater than 30% of a baseline value. In one embodiment, the microbubble comprises a polylactide polymer and nitrogen gas. In one embodiment, the microbubble has an internal diameter ranging from 0.5 to 10 μm. In one embodiment, the conjugation of liposomes onto to the bubble surface is via avidin-biotin bridging. In one embodiment, the polymer microbubble is coated with an albumin. In one embodiment, the first ultrasonic pulse has an acoustic pressure selected from the group consisting of at least 500 kPa, 500 to 1500 kPa, 900 to 1100 kPa and 1000 kPa. In one embodiment, the second ultrasonic pulse has an acoustic pressure selected from the groups consisting of less than 250 kPa, 150-200 kPa, 170-180 kPa and 174 kPa. In one embodiment, the combined ultrasound wave has a frequency selected from the group consisting of 0.5 MHz to 1.0 MHZ, 0.75 MHz to 1.25 MHz, 0.9 MHz to 1.1 MHz, 1.0 MHz to 10.0 MHz, and 1.0 MHz. In one embodiment, the combined ultrasonic wave has a cycle number selected from the group consisting of 5 to 25 cycles, at least 25 cycles, 25 to 1000 cycles, 50 to 150 cycles and 95 to 105 cycles. In one embodiment, the diseased tissue comprises a cancer tissue including, but not limited to, cardiac cancer, bladder cancer, breast cancer, colon cancer, rectal cancer, endometrial cancer, kidney cancer, leukemia, liver cancer, lung cancer, melanoma, sarcoma, non-Hodgkin lymphoma, pancreatic cancer, prostate cancer or thyroid cancer. In one embodiment, the drug comprises a chemotherapeutic drug including, but not limited to, cyclophosphamide, mechlorethamine, chlorambucil, melphalan, dacarbazine, nitrosoureas, temozolomide, daunorubicin, doxorubicin, epirubicin, idarubicin, mitoxantrone, valrubicin, paclitaxel, docetaxel, abraxane, taxotere, epothilone, vorinostat, romidepsin, irinotecan, topotecan, etoposide, teniposide, tafluposide, bortezomib, erlotinib, gefitinib, imatinib, vemurafenib, vismodegib, azacitidine, azathioprine, capecitabine, cytarabine, doxifluridine, fluorouracil, gemcitabine, hydroxyurea, mercaptopurine, methotrexate, tioguanine (formerly Thioguanine), bleomycin, actinomycin, carboplatin, cisplatin, oxaliplatin, tretinoin, alitretinoin, bexarotene, vinblastine, vincristine, vindesine, vinorelbine. In one embodiment, the drug includes but is not limited to, anesthetic or analgesic drugs, muscle relaxant drugs, nerve agent drugs, antihypertensive drugs, heart rate drugs, antiplatelet drugs and/or antithrombin drugs. In one embodiment, the diseased tissue includes, but is not limited to, muscular system tissues, nervous system tissues (e.g., sensory organs, brain, central nervous tissues or peripheral nervous tissues), digestive system tissues, respiratory system tissues, urinary system tissues, reproductive organ tissues (e.g., male or female), endocrine system tissues, circulatory system tissues (e.g., cardiac or vascular), integumenary system tissues and/or lymphatic system tissues. In one embodiment, the non-diseased tissue includes, but is not limited to, muscular system tissues, nervous system tissues (e.g., sensory organs, brain, central nervous tissues or peripheral nervous tissues), digestive system tissues, respiratory system tissues, urinary system tissues, reproductive organ tissues (e.g., male or female), endocrine system tissues, circulatory system tissues (e.g., cardiac or vascular), integumenary system tissues and/or lymphatic system tissues.

### DEFINITIONS

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

The term “cytotoxicity” as used herein, refers to a reduction in cell viability or proliferation as a result of contact with an exogenous compound or mechanical insult. Cytotoxicity is measured by in vitro assays including, but not limited to, caspase activity and cell stains (hematoxylin & eosin or alamarBlue). Cytotoxicity may or may not be associated with reduction in a reduction of tissue or organ function of an otherwise viable tissue.

The term “tissue function” as used herein, refers to an ability of a tissue or organ to successfully perform it innate biological purpose and/or activity. The successful performance of a tissue function can be determined by measuring one or more related biological parameter. For example, “cardiac function” can be determined by measuring one or more cardiac function parameters including, but not limited to, ejection fraction, fractional shortening, left ventricular systolic function and left ventricular mass.

The term “clinical impairment’ as used herein, refers to a reduction of a tissue or organ function to the extent that supportive medical intervention is necessary.

The term “ultrasonic wave” as used herein, refers to a quantitatively defined emission of energy comprising one or more ultrasonic pulses from an ultrasonic transducer having a first specific time period and cycle number (e.g., pulse duration). Each wave cycle may have an intervening second specific time period between each ultrasonic pulse. Each ultrasonic pulse may have the same or different energy emission parameters.

The term “ultrasonic pulse” as used herein, refers to a defined region of an ultrasonic wave having specific quantitative energy emission parameters. For example, these energy emission parameters include, but are not limited to, frequency, acoustic pressure, or cycle number or pulse duration, and pulse repetition frequency. For example, a frequency of an ultrasonic pulse, includes, but is not limited to, 0.75 MHz to 1.25 MHz, 0.9 MHz to 1.1 MHz and 1.0 MHz.

The term “cycle number” as used herein, refers to the number of acoustic cycles that an ultrasonic wave is generated by an ultrasonic transducer. For example, the cycle number includes, but is not limited to, 5 to 25 cycles, at least 25 cycles, 25 to 1000 cycles, 50 to 150 cycles and 95 to 105 cycles.

The term “acoustic pressure” as used herein, refers to a local pressure deviation from the ambient (average or equilibrium) atmospheric pressure, caused by a sound wave (e.g., an ultrasonic wave). The standard unit of sound pressure is the pascal (Pa). As exemplified herein: i) a first ultrasonic pulse is defined as having a high acoustic pressure including, but not limited to, at least 500 kPa, 500 to 1500 kPa, 900 to 1100 kPa and 1000 kPa; and ii) a second ultrasonic pulse is defined as having a low acoustic pressure including, but not limited to, less than 250 kPa, 150-200 kPa, 170-180 kPa and 174 kPa.

The term “exposing” as used herein, refers to the delivery of an ultrasonic wave from an ultrasonic transducer to target tissue region where the transducer is positioned in a line of sight to a target tissue, organ or tumor such that the ultrasonic wave passes around and through the target tissue, organ or tumor. Such exposing is not limited to the target tissue, organ or tumor but also includes the surrounding interstitial spaces and circulatory vasculature.

The term “sonoporation” as used herein, is related to an effect of ultrasound+MB on endothelial barrier function, including, but not limited to: (1) creation of a through-and-through membrane breach (i.e., resembling a bullet hole) through the endothelial cell; and (2) and increased number, or size, of gaps between endothelial cells resulting in enhanced paracellular permeability.

The term “concurrent” or “concurrently” as used herein, refers to the simultaneous intravenous administration of a drug delivery platform (e.g., DoxLPX) and exposure of a target tissue, organ or tumor to an ultrasonic wave.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index, and it can be expressed as the ratio LD50/ED50. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “fibrosis” as used herein, refers to any medical condition marked by increase of interstitial fibrous tissue.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “diseased tissue” as used herein, includes, but is not limited to, muscular system tissues, nervous system tissues (e.g., sensory organs, brain, central nervous tissues or peripheral nervous tissues), digestive system tissues, respiratory system tissues, urinary system tissues, reproductive organ tissues (e.g., male or female), endocrine system tissues, circulatory system tissues (e.g., cardiac or vascular), integumenary system tissues and/or lymphatic system tissues.

The term “non-diseased tissue” includes, but is not limited to, muscular system tissues, nervous system tissues (e.g., sensory organs, brain, central nervous tissues or peripheral nervous tissues), digestive system tissues, respiratory system tissues, urinary system tissues, reproductive organ tissues (e.g., male or female), endocrine system tissues, circulatory system tissues (e.g., cardiac or vascular), integumenary system tissues and/or lymphatic system tissues.

The term “cancer” as used herein refers to a medical condition characterized by unregulated cell proliferation, usually resulting in a cell mass (eg., a tumor). Such tumors may be solid or amorphous. Cancers contemplated in the present invention include, but are not limited to, cardiac cancer, bladder cancer, breast cancer, colon cancer, rectal cancer, endometrial cancer, kidney cancer, leukemia, liver cancer, lung cancer, melanoma, sarcoma, non-Hodgkin lymphoma, pancreatic cancer, prostate cancer or thyroid cancer.

The term “chemotherapy” or “chemotherapeutic” as used herein, refers to the treatment of disease by the use of chemical substances, especially the treatment of cancer by cytotoxic and other drugs. Chemotherapeutic treatment generally uses drugs to stop the growth of cancer cells, either by killing the cells or by stopping them from dividing. Chemotherapy may be given by mouth, injection, or infusion, or on the skin, depending on the type and stage of the cancer being treated. It may be given alone or with other treatments, such as surgery, radiation therapy, or biologic therapy.

The term “chemotherapeutic drugs or compounds” as used herein, refers to specific chemical substances that improve cancer symptoms, such as reducing a tumor volume. Such chemotherapeutic drugs or compounds include but are not limited to, cyclophosphamide, mechlorethamine, chlorambucil, melphalan, dacarbazine, nitrosoureas, temozolomide, daunorubicin, doxorubicin, epirubicin, idarubicin, mitoxantrone, valrubicin, paclitaxel, docetaxel, abraxane, taxotere, epothilone, vorinostat, romidepsin, irinotecan, topotecan, etoposide, teniposide, tafluposide, bortezomib, erlotinib, gefitinib, imatinib, vemurafenib, vismodegib, azacitidine, azathioprine, capecitabine, cytarabine, doxifluridine, fluorouracil, gemcitabine, hydroxyurea, mercaptopurine, methotrexate, tioguanine (formerly Thioguanine), bleomycin, actinomycin, carboplatin, cisplatin, oxaliplatin, tretinoin, alitretinoin, bexarotene, vinblastine, vincristine, vindesine, vinorelbine.

The term “anesthetic drugs” or “analgesic drugs” as used herein, refer to any drug that cause sedation and/or relief of pain. Such anesthetics may include, but are not limited to: i) barbiturates such as amobarbital (Amytal), methohexital (Brevital), thiamylal (Surital), thiopental (Penthothal): ii) benzodiazepines such as diazepam, lorazepam, midazolam; and ii) other compounds such as etomidate, ketamine or propofol. Alternatively, opiod compounds may include but are not limited to alfentanil, fentanyl, remifentanil, sufentanil, buprenorphine, Butorphanol, diamorphine, hydromorphone, levorphanol, pethidine (meperidine), methadone, morphine, nalbuphine, oxycodone, oxymorphone or pentazocine.

The term “muscle relaxant drugs” as used herein, refer to any drug that, at least partially, paralyzes skeletal muscle. Such muscle relaxant drugs may include, but are not limited to succinylcholine, decamethonium, mivacurium, rapacuronium, atracurium, cisatracurium, rocuronium, vecuronium, alcuronium, doxacurium, gallamine, metocurine, pancuronium, pipecuronium or tubocurarine.

The term “nerve agent drugs” as used herein, refer to any drug that, at least partially, inhibits the toxic effects of nerve agents (e.g., organophosphates). Such nerve agent drugs may include, but are not limited to atropine and pralidoxime.

The term “antihypertensive drugs” as used herein, refer to any drug that, at least partially, modulates the regulation of blood pressure, synchrony. For example, antihypertensive drugs may include but are not limited to diuretics, calcium channel blockers, angiotensin converting enzyme inhibitors, angiotensin II receptor antagonists, adrenergic receptor antagonists, vasodilators, renin inhibitors, aldosterone receptor antagonists, alpha-2 adrenergic receptor agonists or endothelium receptor blockers.

The term, “heart rate drugs” as used herein, refers to any drug that, at least partially, modulates the regulation of heart rate. For example, heart rate drugs may include, but are not limited to, epinephrine, norepinephrine, thyroid hormone, caffeine, nicotine or calcium.

The term “antiplatelets” or “antiplatelet drug” as used herein, refers to any drug that prevents aggregation of platelets or fibrin formation (i.e., for example as a prior event to adhesion formation). For example, an antiplatelet drug comprises an inhibitor of glycoprotein IIb/IIIa (GPIIb/IIIa). Further a GPIIb/IIIa inhibitor includes, but is not limited to, xemilofiban, abciximab (ReoPro®) cromafiban, elarofiban, orbofiban, roxifiban, sibrafiban, RPR 109891, tirofiban (Aggrastat®), eptifibatide (Integrilin®), UR-4033, UR-3216 or UR-2922.

The term, “antithrombins” or “antithrombin drug” as used herein, refers to any drug that inhibits or reduces thrombi formation and include, but are not limited to, bivalirudin, ximelagatran, hirudin, hirulog, argatroban, inogatran, efegatran, or thrombomodulin.

The term, “anticoagulants” or “anticoagulant drug” as used herein, refers to any drug that inhibits the blood coagulation cascade. A typical anticoagulant comprises heparin, including but not limited to, low molecular weight heparin (LMWH) or unfractionated heparin (UFH). Other anticoagulants include, but are not limited to, tinzaparin, certoparin, parnaparin, nadroparin, ardeparin, enoxaparin, reviparin or dalteparin. Specific inhibitors of the blood coagulation cascade include, but are not limited to, Factor Xa (FXa) inhibitors (i.e., for example, fondaparinux), Factor IXa (FIXa) inhibitors, Factor XIIIa (FXIIIa) inhibitors, and Factor VIIa (FVIIa) inhibitors.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc. For example, such administering comprises a intravenous infusion or injection of a drug-liposomal microbubble complex into the systemic vasculture.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, saline, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term “biologically active” refers to any molecule having structural, regulatory or biochemical functions. For example, biological activity may be determined, for example, by restoration of wild-type growth in cells lacking protein activity. Cells lacking protein activity may be produced by many methods (i.e., for example, point mutation and frame-shift mutation). Complementation is achieved by transfecting cells which lack protein activity with an expression vector which expresses the protein, a derivative thereof, or a portion thereof.

The term “monodisperse particles” as used herein, include a population of particles wherein at least about 60% of the particles in the population, more preferably 75% to 90% of the particles in the population, or any integer in between this range, fall within a specified particle size range. A population of monodispersed particles deviate less than 10% rms (root-mean-square) in diameter and preferably less than 5% rms.

### BRIEF DESCRIPTION OF THE FIGURES

The file of this patent contains at least one drawing executed in color. Copies of this patent with color drawings will be provided by the Patent and Trademark Office upon request and payment of the necessary fee.

FIG. 1 presents an illustrative set-up for in vivo mouse tumor treatments.
FIG. 1A: Each of the six (6) treatments were administered as 360 μL of the following formulations: i) liposomal doxorubicin with unconjugated polymer microbubbles (MB+LDox; 1.7×10⁸); ii) liposomal doxorubicin conjugated to polymer microbubbles (DoxLPX; 1.7×10⁸); iii) empty liposomes conjugated to polymer microbubbles (ELPX; 1.7×10⁸); iv) liposomal doxorubicin (LDox; 100 μg Dox); v) Dox; (100 μg Dox);; and vi) saline;
(B) A representative schematic diagram depicting the placement of a cannula in the jugular vein, followed by formulation infusion and ultrasound treatment. i.v. administration was performed every 3.5 days, 4 times total. Custom designed complex US Pulse 1 was concurrently delivered during each formulation infusion aimed at the tumor. Echocardiographic imaging was performed on survival mice every 7 days from day 0.

FIG. 2 presents a representative characterization of DoxLPX.
(A) Schematic structure of DoxLPX.
(B) Brightfield and fluorescence microscopic images of DoxLPX.
(C) Size distribution profile of polymer microbubbles (MB) versus DoxLPX.
(D) In vitro Dox release kinetics from DoxLPX exposed to: ii) a combined high and low acoustic pressure US Pulse 2; i) a high acoustic pressure US Pulse 2; and iii) a low acoustic pressure US Pulse 3; and

FIG. 3 presents exemplary data showing MCA205 cancer cell viability and cell proliferation after treatments with the various treatments (n=3). Cancer cells were suspended and pulsed with the US. LDox and DoxLPX=8 μg/mL Dox. MB, ELPX or DoxLPX to cell ratio=136/1.
FIG. 3A: Cell viability 0.5 h after the treatments.
FIG. 3B: Cell viability time course measured at 72 h after treatments.

FIG. 4 presents exemplary data showing that DoxLPX+US has superior efficacy against MCA205 tumor than the other Dox formulations.
(A) Tumor growth plots showing absolute tumor volume as a function of time.
(B) Kaplan-Meier survival curve. DoxLPX treated mice survived longer than Dox-treated or LDox-treated mice. *p<0.05, **p<0.01.

FIG. 5 presents exemplary data showing a protective effect on Left Ventricle (LV) function in mice treated with DoxLPX+US vs. free Dox.
FIGS. 5A-C: Normalized ejection fraction (EF), fractional shortening (FS) and LV mass index, *p<0.05, **p<0.01 vs. Day 0 data;
FIGS. 5D-F: Incidence of animals with varying severity of EF decrease, FS decrease, and LV mass index increase on Day 21 vs. Day 0. (n=8-9). Free Dox was associated with the highest incidence. *p<0.05 Fisher's exact test.

FIG. 6 presents exemplary data showing representative microscopic images (20×) of H&E stained heart tissues of MCA205 tumor-bearing mice post treatment. Saline and ELPX+US and treatments are shown as control groups. Dox, LDox, DoxLPX+US, LDox+MB+US (5 mg/kg equiv. Dox, 4 treatments in 14 days, accumulative dosage 20 mg/kg, n=8-9). The black arrows point to cardiomyocyte vacuolization.

FIG. 7 presents exemplary data showing that DoxLPX+US treatment protects from Dox-induced cardiac remodeling.
FIG. 7A: Representative cardiac sections stained with Sirius red/fast green to detect interstitial fibrosis.
FIG. 7B: Quantification of fibrosis area in heart tissue by morphometry in ventricular cross-sections.; n=8-9 per group, data shown as mean±SD. Red, collagen fibrosis; Green, non-collagen components.

FIG. 8 presents exemplary data showing that free Dox causes more apoptosis, as measured by caspase-3 activity, in heart tissue homogenate vs. other Dox treatments. n=8-9, *p<0.05, **p<0.01.

FIG. 9 presents exemplary data showing representative ex vivo fluorescence images showing biodistribution of a Dox analog (Cy5.5).
FIG. 9A: Representative ex vivo fluorescence images of organs excised 3.5 hrs after treatment, demonstrating higher intra-tumoral and lower cardiac uptake of Cy5.5 after Cy5.5LPX+US (third panel from left).
FIG. 9B: Quantitative biodistribution (n=3) of Cy5.5 concentration in the tumors, blood, heart, and lung in tumor-bearing mice 3.5 h post i.v. injection of free Cy5.5, LCy5.5, LCy5.5+MB+US and Cy5.5 LPX+US.
FIG. 9C: Cy5.5 concentration in the kidney, liver, and spleen.

FIG. 10 presents exemplary Z-average size parameters of liposomal Dox (LDox).

FIG. 11 presents exemplary data showing the effect of the various DOX treatments on MCA 205 tumor growth.
FIG. 11A: A representative 3D reconstruction tumor size and calculated tumor volumes using high-frequency ultrasound scanning in mice receiving ELPX+US, Dox, LDox, DoxLPX+US, MB+LDox+US and saline control treatments.
FIG. 11B: Normalized tumor growth curve of mice in each cohort vs. their initial volume on Day 0;
FIG. 11C: A normalized average tumor volume on Day 14 or Day 17.5 after the 1st treatment. ^{a}: p<0.05 vs. saline group, ^{aa}: p<0.01 vs. saline group, ^{b}: p<0.05 vs. ELPX+US group, ^{bb}: p<0.01 vs. ELPX+US group, *p<0.05, ***p<0.001.

### DETAILED DESCRIPTION OF THE INVENTION

The present invention is directed towards improved methods of treating cancer tumors. In particular, the invention inhibits tumor growth without impairing the function of non-cancerous tissues. For example, a systemically administered and ultrasonically-directed chemotherapeutic-loaded liposome conjugated to a polymer microbubble inhibits tumor growth on a cancerous tissues without impairing the function of non-cancerous tissues.

In summary, treatment with DoxLPX+US showed; i) increased Dox concentration in a tumor; ii) a significant slow-down in tumor growth; and iii) a prolonged median survival time. Both LDox and DoxLPX administration reduced drug extravasation into the myocardium. LDox+MB+US also demonstrated superior tumor growth inhibition as compared to free Dox and LDox. Three weeks after treatments, DoxLPX+US group showed significantly better left ventricular function indices as determined by echocardiography imaging than the free Dox group.

These findings are consistent with biodistribution studies using Cy5.5 as a Dox analog, demonstrating the highest ratio of tumor to heart accumulation after treatment with Cy5.5LPX+US. Concordantly, H&E and Sirius red/fast green staining of the heart tissue showed normal architecture of cardiac myocytes and a significantly less interstitial/perivascular fibrosis in the DoxLPX+US group compared to the free Dox group. Consequently, a DoxLPX formulation in conjunction with ultrasound provides a targeted drug delivery platform having superior anti-tumor efficacy and reduced cardiac function impairment when compared with systemic administration of free Dox or LDox+MB co-administration.

The data presented herein demonstrate that, as compared to free Dox and liposomal Dox, DoxLPX+US treatment inhibited tumor growth and increased survival in sarcoma-bearing mice. As compared to all other Dox-treated groups, DoxLPX+US attenuated the known adverse effects of Dox on cardiac function. For example, these adverse effects on cardiac function included, but were not limited to, systolic dysfunction, left ventricular hypertrophy and myocardial collagen deposition. Biodistribution data substantiated that DoxLPX+US targets Dox delivery to the tumor site, resulting in tumor growth inhibition equivalent to that achieved by free Dox. The reduced adverse cardiac function of DoxLPX treatment is associated with less cardiac tissue Dox accumulation, resulting in preservation of ventricular function. Consequently DoxLPX+US treatment may improve cancer treatment outcome by allowing higher doses of Dox to be administered while avoiding impaired cardiac function.
I. Doxorubicin Cancer Therapy

Doxorubicin (Dox) is an anthracycline that is one of the best-understood and the most widely used chemotherapeutic agents. However, Dox induces cytotoxicity in an irreversible and cumulative dose-dependent manner. Dox dosing is largely governed by the maximal tolerated cumulative dose (400-550 mg/m2). Ewer et al., “Cardiotoxicity of anticancer treatments” Nat Rev Cardiol. 2015; 12(9):547-558. As such, a growing number of cancer survivors are at increased lifetime risk of Dox-induced cardiotoxicity. For example, the onset of Dox-induced cardiotoxicity may be delayed until as many as 10-15 years after the cessation of chemotherapy. Dox-induced cardiomyopathy is strongly linked to an increase in cardiac oxidative stress, as evidenced by reactive oxygen species (ROS), including mitochondrial-dependent ROS, NOS dependent ROS, NAD(P)H dependent ROS, etc. Dox also induces apoptosis of both cardiomyocytes and endothelial cells by caspase activation and internucleosomal DNA degradation. Octavia et al., “Doxorubicin-induced cardiomyopathy: From molecular mechanisms to therapeutic strategies” J Mol Cell Cardiol. 2012; 52(6):1213-1225. Repeated damage to the mitochondria and defenses against free radicals is also believed to contribute to cumulative cardiomyopathy. Ogura et al., “Electron-Spin-Resonance Studies on the Mechanism of Adriamycin-Induced Heart Mitochondrial Damages” Cancer Res. 1991; 51(13):3555-3558. The use of combination regimen with overlapping toxicities is likely to potentiate Dox-induced cardiotoxicity (e.g. cyclophosphamide, trastuzumab, taxanes, and anthracyclines). Rahman et al., “Anthracycline-induced cardiotoxicity and the cardiac-sparing effect of liposomal formulation” Int J Nanomed. 2007; 2(4):567-583.

Pegylated-liposomal Dox (PLD) (e.g. Doxil®, Caelyx®) and non-pegylated liposomal Dox (LDox) (e.g. Myocet®, D-99) formulations represent commercially available formulations of Dox. Rafiyath et al., “Comparison of safety and toxicity of liposomal doxorubicin vs. conventional anthracyclines: a meta-analysis” Exp Hematol Oncol. 2012; 1(1):10. Doxil® and Caelyx® are FDA-approved for treatments of AIDS-related Kaposi's sarcoma and ovarian cancer. Lentacker et al., “Design and Evaluation of Doxorubicin-containing Microbubbles for Ultrasound-triggered Doxorubicin Delivery: Cytotoxicity and Mechanisms Involved” Mol Ther. 2010; 18(1):101-108.

Because of their diameter, intravenously injected LDox formulations cannot escape the vascular space in organs that have tight capillary junctions, such as the myocardium and gastrointestinal tract, while the drug delivery to tumor sites lined by compromised vasculature get enhanced. PLD has a polyethylene glycol (PEG) coating around the liposome bilayer to protect the molecule further from phagocytosis by the mononuclear phagocytes. This also confers a longer half-life, smaller volume of distribution and reduced clearance of the drug, all resulting in a good efficacy. However, PLD did not improve the maximally tolerated dose compared to free Dox and has a preferential concentration in the skin due to the long circulation and some drug leaks from capillaries in the palms of the hands and soles of the feet. This causes a new dose-limiting toxicity syndrome called palmar-plantar erythrodysesthesia (PPE). Lorusso et al., “Pegylated liposomal doxorubicin-related palmar-plantar erythrodysesthesia (‘hand-foot’ syndrome)” Ann Oncol. 2007; 18(7): 1159-1164.

Dox is the standard of care for the treatments of sarcomas and tumor response rate is correlated with the dosage that makes the Dox dose limitations particularly problematic. Duggan et al., “Pegylated Liposomal Doxorubicin A Review of its use in Metastatic Breast Cancer, Ovarian Cancer, Multiple Myeloma and AIDS-Related Kaposi's Sarcoma” Drugs. 2011; 71(18):2531-2558. Soft tissue sarcomas (STS) are malignant tumors originating from extra-skeletal connective tissues that can arise at any site. LDox was found to be as effective as free Dox or has non-inferior efficacy to STS. Spira et al., “The use of chemotherapy in soft-tissue sarcomas” Oncologist. 2002; 7(4):348-359. The dual-edged dose-related therapeutic efficacy and side-effects of Dox in addition to the known benefits of regional therapies define a great need for a local non-invasive Dox delivery platform with low systemic toxicity for the treatment of STS.

The free Dox in plasma is believed responsible for the toxicity of non-tumor related organs. Liposomal Dox has been designed to reduce the cardiotoxicity of Dox while preserving its antitumor efficacy. The LDox used in this study is a similar biotinylated counterpart of Doxil®. It has been reported that 240 mg/mm². Doxil® clinically reduced the risk of cardiotoxicity occurrence from 26% of the free Dox to 10%, 4 but did not completely spare the cardiotoxicity.

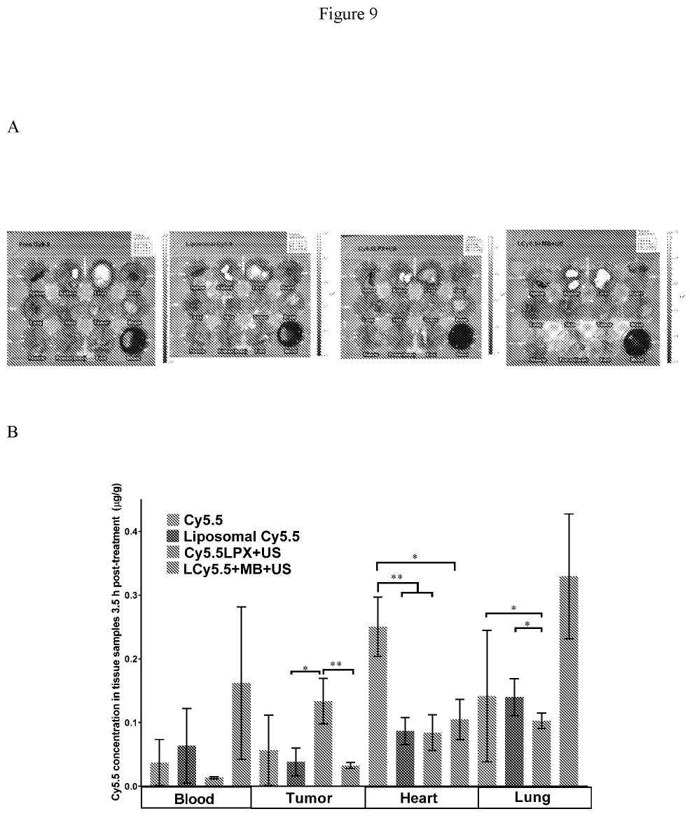

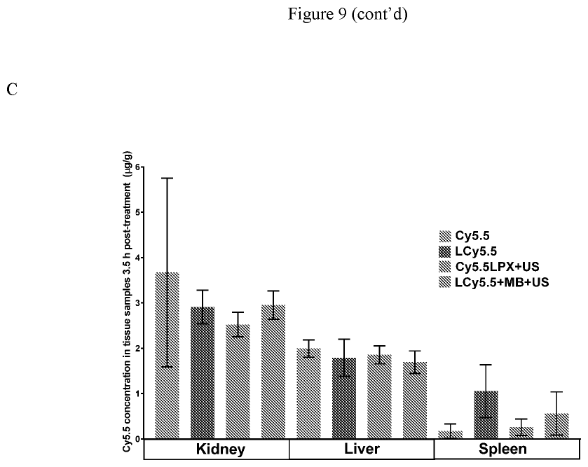

Both liposomal Dox or LPX formulation can prevent the extravasation into the myocardium. But the preferential concentration Doxil® in the skin led to dose-limiting side effects. Following the administration, small amounts of the drug can leak from capillaries in the palms of the hands and soles of the feet. This results in redness, tenderness, pain, and peeling of the skin. LDox+MB+US treatment actively enhanced the LDox permeation in the tumor and antitumor efficacy by UTMC but cannot reduce the circulation time and the accumulation of LDox in the skin. The biodistribution and cardiac function data presented herein indicate DoxLPX+US treatment showed a much faster clearance in the blood and less accumulation in the heart/lung may prevent the occurrence of PPE and cardiotoxicity. See, FIG. 9.

Resolving Dox dose-limiting syndromes means a possible increase in drug dosage administration, in addition to the enhanced tumor inhibition makes DoxLPX+US as an optimal approach to maximize the chemotherapy efficacy while reducing reductions in tissue and/or organ function. In summary, the data presented herein shows that DoxLPX+US treatment demonstrates a superior tumor inhibition efficacy while being protective of cardiac contractile function, histologic lesion, fibrosis and apoptosis in the heart tissue and faster clearance from the blood circulation that may cause PPE side-effects. The LDox+MB+US administration was observed to have similar advantages.
II. Microbubble Ultrasound Treatment

Microbubbles (MBs) as ultrasound contrast agents are intravenously injectable gas-filled microspheres consisting of a biocompatible shell (lipids, proteins or polymers). Ultrasound-targeted MB cavitation (UTMC), the alternating compression and expansion of MBs under the influence of an ultrasonic field, is an approach for image-guided local delivery of drugs or nucleic acids that enable the triggered unloading of cargo at the region of interest. Yu et al., “Low Intensity Ultrasound Mediated Liposomal Doxorubicin Delivery Using Polymer Microbubbles” Mol Pharmaceut. 2016; 13(1):55-64; and Fan et al., “Folate-conjugated gene-carrying microbubbles with focused ultrasound for concurrent blood-brain barrier, opening and local gene delivery” Biomaterials. 2016; 106:46-57.

UTMC as a drug delivery strategy has advantages in many aspects, including its capability to reach deep tissue in a noninvasiveness manner, local applicability, and lower cost. UTMC can result in increased permeability of vascular walls and cell membrane. UTMC combined with nanoscale LDox is promising because LDox drug efficacy largely depends on its penetration through vascular endothelium and tumor interstitium. UTMC has been shown to enhance the accumulation and permeation of the liposomal drug in tumors. Theek et al., “Sonoporation enhances liposome accumulation and penetration in tumors with low EPR” J Control Release. 2016; 231:77-85.

Doxorubicin-Lipopolycomplex (DoxLPX), a form of LDox conjugated to MBs and was designed as an advanced delivery vehicle to achieve a non-invasive spatially controlled targeted delivery by UTMC. Deng et al., “Reversal of multidrug resistance phenotype in human breast cancer cells using doxorubicin-liposome-microbubble complexes assisted by ultrasound” J Control Release. 2014; 174:109-116; Geers et al., “Self-assembled liposome-loaded microbubbles: The missing link for safe and efficient ultrasound triggered drug-delivery” J Control Release. 2011; 152(2):249-256; Ferrara et al., “Ultrasound microbubble contrast agents: Fundamentals and application to gene and drug delivery” Annu Rev Biomed Eng. 2007; 9:415-447; and Eisenbrey et al., “Development and optimization of a doxorubicin loaded poly(lactic acid) contrast agent for ultrasound directed drug delivery” J Control Release. 2010; 143(1):38-44.

In in vitro studies, DoxLPX and ultrasound (US) treatment killed cancer cells by the local release of free Dox as well as enhancement of the cellular drug uptake via MB cavitation that perforates the cell membranes. Compared to the DoxLPX, the co-injection of LDox and MB cannot prevent the undesired LDox extravasation and accumulation in the undesired tissues, e.g. skin capillaries. The prior studies showed similar systems achieved the US-triggered drug release and sonoporation, but did not provide detailed in vivo drug biodistribution, antitumor efficacy, and cardiac toxicity information. Lentacker et al., “Design and Evaluation of Doxorubicin-containing Microbubbles for Ultrasound-triggered Doxorubicin Delivery: Cytotoxicity and Mechanisms Involved” Mol Ther. 2010; 18(1):101-108, Yu et al., “Low Intensity Ultrasound Mediated Liposomal Doxorubicin Delivery Using Polymer Microbubbles” Mol Pharmaceut. 2016; 13(1):55-64; and Lin et al., “Enhancement of focused ultrasound with microbubbles on the treatments of anticancer nanodrug in mouse tumors” Nanomed-Nanotechnol. 2012; 8(6):900-907.

The data presented herein systematically analyzes the local delivery and antitumor efficacy of DoxLPX and LDox+MB co-administration with therapeutic US and their effect on cardiac function.

In one embodiment, the present invention contemplates a US-mediated drug delivery platform that can achieve a targeted drug delivery, improve drug permeability in tumors, inhibit tumor growth, and alleviate impaired tissue function. In one embodiment, MBs comprise a gas core (˜3-μm diameter) stabilized by a biocompatible and biodegradable PLA shell. See, FIG. 2A. In one embodiment, the MBs comprise at least one polymer (e.g., poly-lactic acid PLA) which is more stable than a lipid microbubble. In one embodiment, the MBs comprise human serum albumin (HSA) crosslinked and coated on the polymer shell. Although it is not necessary to understand the mechanism of an invention, it is believed that an HAS-coated MB minimizes immunogenicity and provides reactive thiol groups as a conjugate partner for a liposome. The average diameter of the DoxLPX complex increased from 3.26 μm to 3.52 μm after LDox conjugation to the MB. See, FIG. 2C. The DoxLPX complex is believed to have a Dox loading content that is higher than any previously reported for any nanoparticle. Lentacker et al., “Design and Evaluation of Doxorubicin-containing Microbubbles for Ultrasound-triggered Doxorubicin Delivery: Cytotoxicity and Mechanisms Involved” Mol Ther. 2010; 18(1):101-108; Yu et al., “Low Intensity Ultrasound Mediated Liposomal Doxorubicin Delivery Using Polymer Microbubbles” Mol Pharmaceut. 2016; 13(1):55-64; Deng et al., “Reversal of multidrug resistance phenotype in human breast cancer cells using doxorubicin-liposome-microbubble complexes assisted by ultrasound” J Control Release. 2014; 174:109-116; and Lin et al., “Enhancement of focused ultrasound with microbubbles on the treatments of anticancer nanodrug in mouse tumors” Nanomed-Nanotechnol. 2012; 8(6):900-907.

In one embodiment, the present invention contemplates a method comprising administering a dose of Dox (e.g., 40 mg for an adult of about 80 kg) that can be delivered by about 6.78×10¹⁰ DoxLPX microbubbles. In comparison, this dosage requires an 18-fold higher number of LDox liposomes (e.g., 1.23×10¹²). Lentacker et al., “Design and Evaluation of Doxorubicin-containing Microbubbles for Ultrasound-triggered Doxorubicin Delivery: Cytotoxicity and Mechanisms Involved” Mol Ther. 2010; 18(1):101-108. Although it is not necessary to understand the mechanism of an invention, it is believed that this improved DoxLPX drug loading capacity has the potential to minimize the risk of an immune response. Such immune responses may come from a nanocarrier and/or a volume of the gas escaped from the LPX after UTMC that may block vessels, displace tissues, or trigger an inflammatory cascade.

It has been suggested that US pulses between 1-10 MHz can enhance the intracellular delivery and extravasation of microbubble-drug complexes circulating through the vasculature.

Theek et al., “Sonoporation enhances liposome accumulation and penetration in tumors with low EPR” J Control Release. 2016; 231:77-85; and Helfield et al., “Biophysical insight into mechanisms of sonoporation” P Natl Acad Sci USA. 2016; 113(36):9983-9988. In one embodiment, the US pulse is a low acoustic pressure US pulse (˜170 kPa). In one embodiment, the US pulse is a high acoustic pressure US pulse (˜1 MPa). Although it is not necessary to understand the mechanism of an invention it is believed that a low acoustic pressure pulse releases more Dox from an DoxLPX complex in 60 s than a high acoustic pressure US pulse. It is further believed that destruction of a DoxLPX complex quickly occurs when exposed to a high acoustic pressure US pulse thereby releasing core gas from the polymer shell under high pressure.

By echocardiographic observation (data not shown), MB passage through the tumor vasculature takes about 5˜15 s, depends on the tumor size and structure of the vasculature. The LPX particle may enter the tumor vasculature multiple times in the blood circulation and release the Dox during the treatment time. It has previously been demonstrated that low acoustic pressure US pulses (<200 kPa) enhance MB tissue uptake by stimulating endocytosis, while high acoustic pressure US pulses (>400 kPa) lead to sonoporation on the cell membrane. De Cock et al., “Ultrasound and microbubble mediated drug delivery: Acoustic pressure as determinant for uptake via membrane pores or endocytosis” J Control Release. 2015; 197:20-28. In one embodiment, the present invention contemplates a method comprising a high acoustic pressure working cycle. In one embodiment, the high acoustic pressure working cycle enhances drug permeability through a tumor vasculature. Although it is not necessary to understand the mechanism of an invention, it is believed that exposing a tissue to high acoustic pressure US for a prolonged time period results in toxicity and generates cell debris.

In one embodiment, the present invention contemplates a method comprising administering a high acoustic pressure US pulse immediately followed by a low pressure acoustic pressure US pulse concurrently with a drug-LPX complex infusion. The data presented herein shows that this US regimen achieves an immediate and sustained drug release that provides an unpredicted and superior drug release profile as compared to either a high acoustic pressure US pulse or a low acoustic pressure pulse when given alone. See, FIG. 2D. Although it is not necessary to understand the mechanism of an invention, it is believed that a combined high/low acoustic pressure US pulse results in a sustained LPX US-responsiveness and an effective sonoporation through an endothelium wall.
III. Ultrasound Acoustic Pressure Microbubble Drug Delivery

The data herein presents DoxLPX complexes comprising a polymer microbubble coated with crosslinked human serum albumin (HSA) and conjugated liposomal Dox. DoxLPX complexes were intravenously injected into MCA205 sarcoma bearing mice, and concurrently, therapeutic ultrasound was delivered to the tumor site. The data compared equivalent dosages of free Dox, LDox, and LDox+MB co-administration with ultrasound treatment (e.g., LDox+MB+US). Tumor growth and cardiac function were serially recorded with ultrasound imaging. Histology analysis of the heart tissue and apoptosis assay were used to analyze the causes of changes in cardiac function.

A. Characterization of LDox and DoxLPX

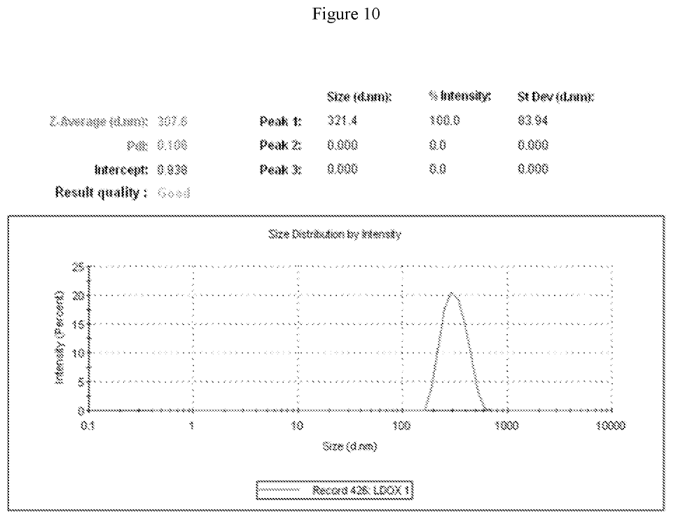

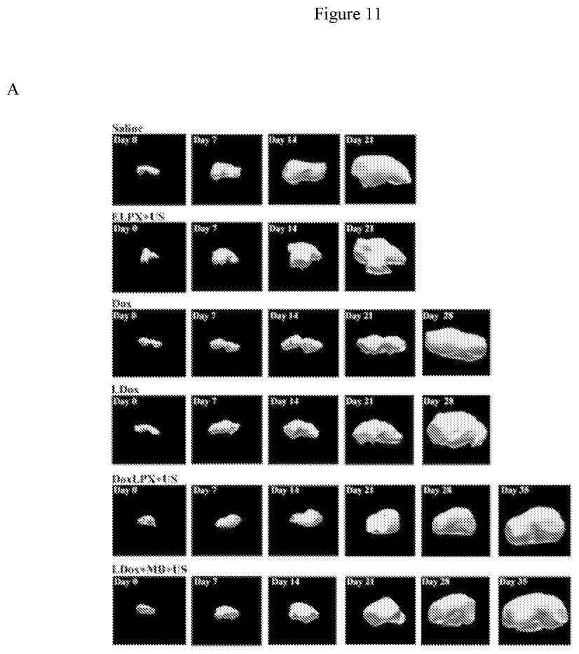

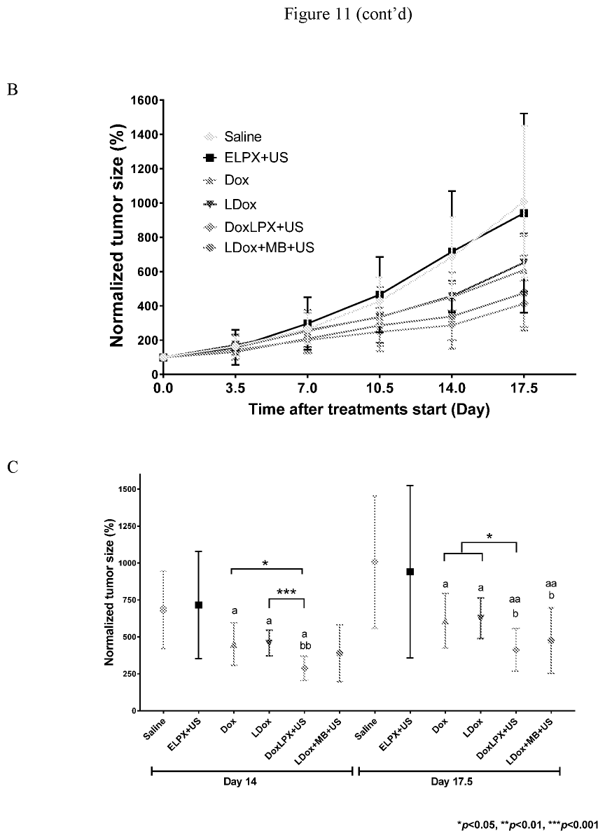

Liposomal Dox (Ldox) particles had a z-average size of 307.6 nm and a polydispersity index of 0.106. (See, FIG. 10). After conjugation of LDox to the polymer MB, the resulting DoxLPX complex demonstrated red fluorescence and a larger diameter compared to the polymer

MB (3.52±1.17 μm vs. 3.26±1.05 μm of polymer MB, consistent with Dox loading on the MB surface. See FIGS. 2A, 2B and 2C), A fluorescence dequenching assay using 0.3% Triton X-100, showed that the magnitude of Dox loading on DoxLPX was 0.53-0.59 μg Dox per 1×10⁶ DoxLPX complexes.

DoxLPX complex release profiles of Dox were measured over a 60 s period of exposure to each of three (3) acoustic pulses. DoxLPX did not release free Dox in the absence of US. The high-pressure US regime caused more rapid Dox release (Pulse 2: 17.0% release in 10 sec) as compared to the low-pressure regime (Pulse 1: 9.1% release in 10 sec). However, after about ten (10) seconds no further DOX release with Pulse 2, likely due to complete MB destruction. In contrast, the low-pressure Pulse 1 induced a long-term Dox release from DoxLPX. For example, approximately 23.9% DOX release was observed after sixty (60) seconds of Pulse 1 exposure. Surprisingly, when the high pressure Pulse 2 was immediately followed by the low acoustic pressure Pulse 1 (e.g., depicted as Pulse 3) Dox was released from DoxLPX having a rapid and sustained release profile (e.g., 13.7% at 10 sec, 24.9% at 60 s). See, Table 1 and FIG. 2D.

TABLE 1
Ultrasound pulse configurations (1 MHz) in the in vitro study

| Pulse | | Duty | I_{SPTA} |
|---|---|---|---|
| name | Pulse configuration | cycle (%) | (W/cm²) |
| Pulse 1 | 170 kPa (500 μs) followed by 500 μs off | 50% | 0.48 |
| Pulse 2 | 1 MPa (10 μs) followed by 990 μs off | 1% | 0.33 |
| Pulse 3 | 1 MPa (10 μs) + 170 kPa (490 μs) | 50% | 0.80 |
| | followed by 500 μs off | | |

US acoustic pressure-mediated drug release was previously reported using high pressure treatment pulses (H) of: i) fixed peak negative pressure of 1500 kPa at five cycles per minute (H1500-5), 643 cycles per minute (H1500-643), and 2000 cycles per minute (H1500-2k). Low pressure treatment pulses (L) were also reported of 50,000 cycles per minute at a pressure of 170 kPa (L170-50k) and 300 kPa (L300-50k). Yu et al., Mol Pharm 13:55-64 (2015). The pulse repetition frequency was set at 10 Hz. Parameters were chosen to result in “high” and “low” pressure configurations with equivalent spatial peak temporal average intensity (ISPTA) values.

These US regimens reported by Yu et al. are not predictive of those disclosed herein because they differ in both acoustic pressures as well as cycle numbers and total durations of exposure. Furthermore, this reference is limited to the use of high and low acoustic pressure US treatments that are delivered separately, as opposed to a combination of high and low pulses (e.g., Pulse 3) as disclosed herein.

B. In Vitro Cytotoxicity of DoxLPX and LDox+MB Co-Injection

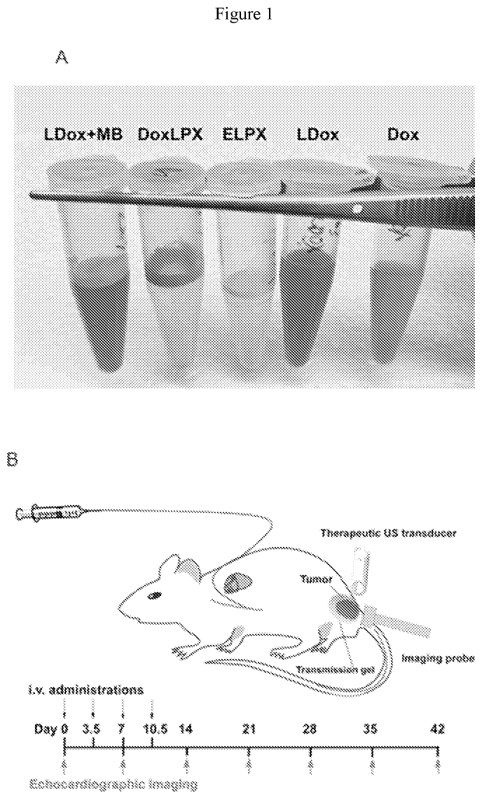

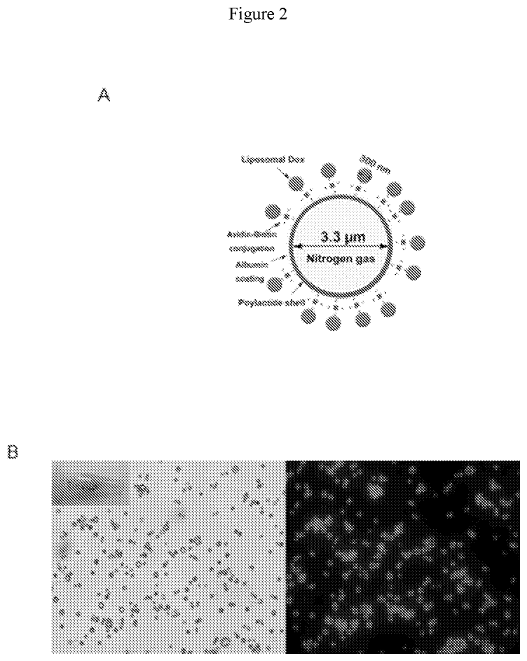

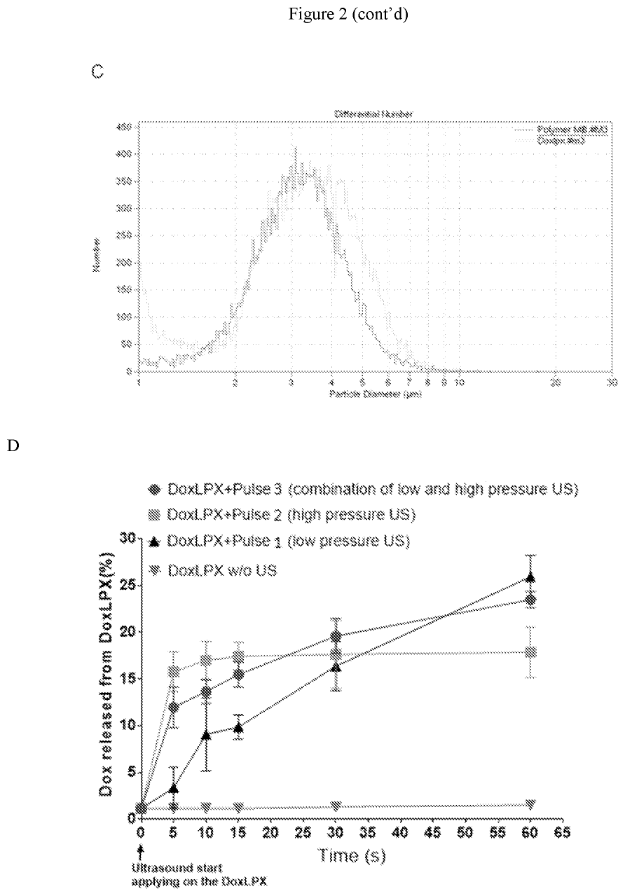

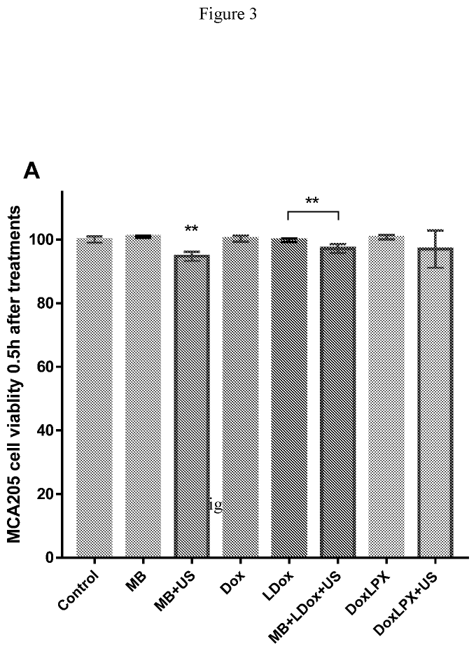

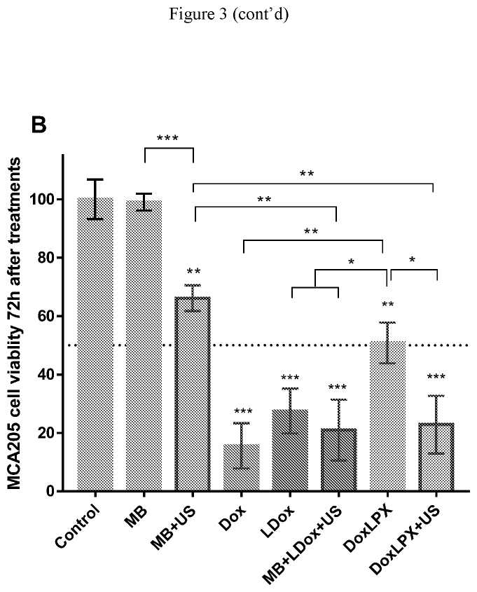

Cytotoxicity was determined by assessing the viability of MCA205 cells following 30 minutes and 24, 48 and 72 hours after treatment with the various DOX formulations. See, FIG. 3A and 3B. The data show that the control ELPX treatment was not cytotoxic at any time point. While the ELPX+US treatment did not cause cell death at 30 minutes, the number of viable MCA206 cells was significantly lower (about 65˜70% of untreated control or ELPX alone) in the following 3 days. See, FIG. 3A. These data suggest that while a thirty (30) minute US-MB exposure (e.g., without drug) does result in cell death, there may be a general effect on cell proliferation. Although it is not necessary to understand the mechanims of an invention, this delayed effect from US+MB can also be due in part to a more protracted cell dealth response (eg from apoptosis) than detected at 30 minutes, in addition to general effect on cell proliferation. Regardless, there is an effect of the ultrasound-microbubble interaction itself on the number of viable cells, although this may or may not translate into tumor size differences in vivo.

Free Dox treatment was more cytotoxic (p<0.05) than LDox against cells at both the 24 h and 48 h time points, but not at 72 hours. See, FIG. 3C. These data suggest that there may be a delayed bioavailability of Dox when encapsulated in liposomes.

LDox+MB+US treatment significantly decreased cell viability and/or proliferation at 0.5 h and 24 h when compared to the LDox formulation. See, FIG. 3A. No significant difference in cytotoxicity was observed between LDox+MB+US and LDox treatments at 48 h and 72 h, likely because LDox cytotoxicity may be a factor influencing cell viability. Free Dox and Ldox treatments showed more cytotoxicity than DoxLPX in the absence of US at 48 h and 72 h. DoxLPX+US caused significantly less cell viability compared to DoxLPX without US at 48 h and 72 h. See, FIG. 3B.

These data show that DoxLPX without US induce significantly reduced cytotoxicity to cells as compared to free Dox which showed the highest cytotoxicity at 24, 48, and 72 h, because the free Dox enter cells and the nuclei easier and faster by diffusion. US sonication combined with DoxLPX induced a significantly higher toxicity/growth inhibition to MCA205 cells than the non-US treated counterparts. DoxLPX+US achieved the 2nd highest cytotoxicity at 48 h and 72 h in all treatments. This indicates when applied in vivo, DoxLPX can be expected to generate more cytotoxicity in the US-aimed target tissue region (e.g., a tumor tissue) and protect tissue and/or organ function in non-target regions (e.g., a non-tumor tissue).

C. In Vivo Antitumor Efficacy

Fifty-four (54) tumor-bearing mice were randomly divided into six (6) treatment groups to examine the therapeutic significance of US-mediated Dox delivery and related effects on cardiac function. All animals were alive with normal activity and no sign of microbial infection during and after treatments. In the first 3 days post-surgery, mice in all groups lost about 10% body weight due to the traumatic catheter dwelling procedure. The average body weight of mice recovered on day 7 and constantly gained the weight (data not shown). Although surgery moderately traumatized the mice, the i.v. administration of treatments through an indwelling catheter is still the preferable route because: (1) i.v. administration via tail vein led to high shear stress that can partially break the MBs in the formulation; (2) tail vein injection to adult C57BL/6J mice is difficult due to the pigment on the tails.

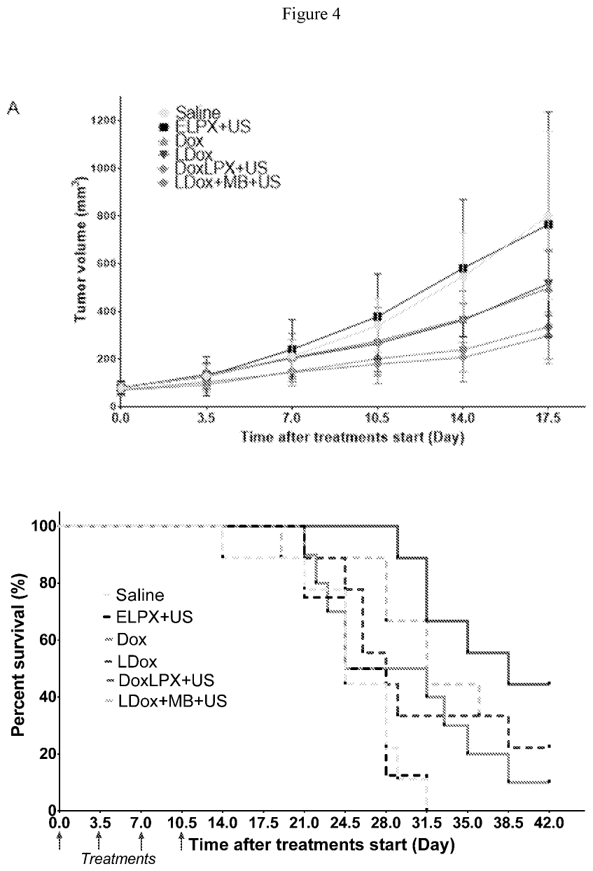

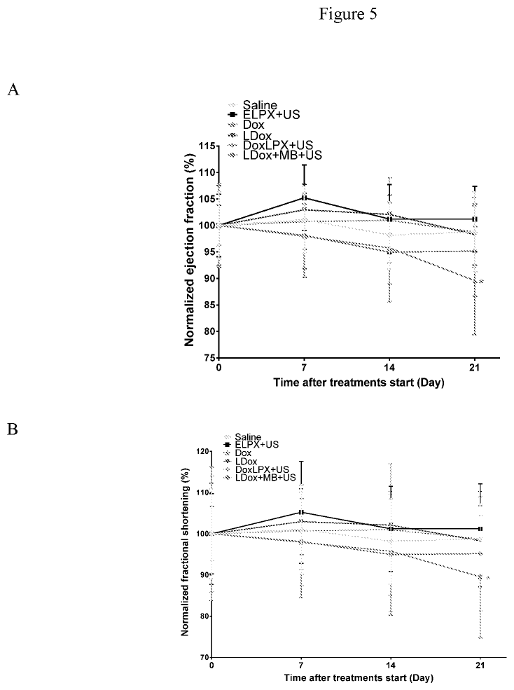

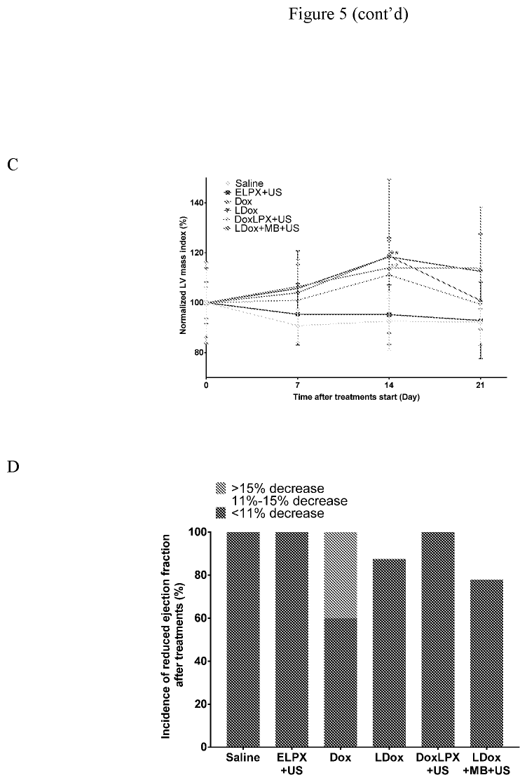

Tumor size measurement was based on 3D reconstructed high-resolution US imaging. Tumor growth was effectively hindered and the normalized tumor size in DoxLPX+US treated group are significantly smaller than that of Dox and LDox group on day 14 and day 17. See, FIG. 5A and S3. A significant slow-down of tumor growth translated in a prolongation of median survival of 55% and 29% for DoxLPX+US and LDox+MB+US, respectively, as compared to controls. See, Table 2.

TABLE 2
Median survival time and tumor doubling time (DT) of the mice after treated with
various formulations.

| | Saline | ELPX + US | Dox | LDox | DoxLPX + US | LDox + MB + US |
|---|---|---|---|---|---|---|
| Median | 24.5 ± 5.2 | 26.3 ± 3.7 | 28.0 ± 7.5 | 28.0 ± 7.9 | 38.5 ± 45.4***^{,###,§} | 31.5 ± 5.8**^{,##} |
| survival time ± | | | | | | |
| standard | | | | | | |
| deviation | | | | | | |
| (Day) | | | | | | |
| Tumor | 5.2 ± 0.5 | 5.7 ± 1.1 | 7.1 ± 1.0**^{,#} | 7.2 ± 0.7***^{,##} | 9.2 ± 2.7**^{,##,§} | 8.7 ± 2.5**^{,##} |
| doubling | | | | | | |
| time (Day) | | | | | | |

*p < 0.05,
**p < 0.01, and
***p < 0.001 vs. saline group;
^{#}p < 0.05,
^{##}p < 0.01, and
^{###}p < 0.001 vs. ELPX + US group;
^{§}p < 0.05 vs. Dox group.

In contrast, free Dox and LDox treatments only prolonged median survival time 14%. ELPX+US treated group showed no difference in the same tumor growth and body weight change as compared to the saline control group. These data indicate that the UTMC of LPX carrier did not induce the significant toxicity or immune response to the tumor or animal.

Tumor doubling time (DT) of DoxLPX+US group is significantly longer than free Dox, ELPX+US group or control. LDox+MB+US group also showed numerically longer DT as compared to the Dox and LDox group. LDox+MB (polymer or lipid) and tumor-targeted UTMC showed enhanced therapeutic responses of chemotherapy drug by disrupting the vascular wall in the sonicated tumor tissues or blood-brain barrier (BBB) in prior studies. Theek et al., “Sonoporation enhances liposome accumulation and penetration in tumors with low EPR” J Control Release. 2016; 231:77-85; Lin et al., “Enhancement of focused ultrasound with microbubbles on the treatments of anticancer nanodrug in mouse tumors” Nanomed-Nanotechnol. 2012; 8(6):900-907; and Kovacs et al., “Prolonged survival upon ultrasound-enhanced doxorubicin delivery in two syngeneic glioblastoma mouse models” J Control Release. 2014; 187:74-82. These data show that US-mediated polymer-based LPX conjugated with a liposomal drug formulation can achieve superior antitumor efficacy in vivo as compared to the same dosage of a free systemic drug and/or a US-mediated LDrug+MB co-administration.

Tumor volumes were measured before and after Dox formulation treatments over approximately 17.5-20 days. Control mice receiving either saline alone or ELPX+US had a rapid exponential tumor growth to 1008% and 941% of original tumor volume by day 17.5, respectively. Treatment with free Dox or LDox resulted in moderate growth inhibition (tumor volumes of 610% and 652% as compared to baseline, respectively) by Day 17.5. In contrast, treatment with DoxLPX+US or LDox+MB co-injection+US resulted in greater tumor growth inhibition (e.g., tumor volumes of 413% and 475% as compared to baseline). See, FIG. 4A.

In the four (4) treatment groups receiving a Dox formulation (˜100 μg equivalent doses of Dox), after Day 14 (e.g., four (4) days after the final dose) tumor growth began to accelerate at a rate similar to that of the negative controls. Comparisons among tumor volumes between Day 14 and Day 17.5 are shown as a function of treatment group. At Days 14 and 17.5, mice receiving any of the four (4) Dox-containing formulations had smaller tumors as compared to negative control mice receiving saline or ELPX+US. While the mean size of tumors after treatment with DoxLPX+US was not statistically different from tumors treated with LDox+MB co-injection, it was only the DoxLPX+US-treated tumors where tumor size reduction reached statistical significance as compared to the free Dox and LDox-treated tumors by Days 14 and 17.5.

Serial ultrasound 3D volume reconstructions of a representative tumor from each experimental group spanning Day 0 up to Day 35 were created. The data show a rapid tumor growth in mice receiving either saline or ELPX+US by Day 14, with the volumetric images indicate clearly larger tumor size as compared to the other treatment groups on the same day.

See, FIG. S3A. The tumors shown for Dox and LDox treated mice (middle two rows) demonstrated slower growth as compared to the controls, but growth accelerated by Day 28. The bottom two panels show tumors from two mice treated with DoxLPX+US and co-injection LDox+MB+US. These data show the slowest tumor growth subsequent to DoxLPX+US treatment (e.g., cf Days 21 to 35 as compared to the Ldox+MB+US co-injection group). Unlike the mice in the other experimental groups, these two mice survived until the end of the protocol.

Treatment-related difference in tumor growth resulted in significant differences in tumor doubling time between the experimental groups. See, Table 2. Tumor doubling time in mice treated with any Dox formulation was significantly more prolonged as compared to that in of control mice receiving either saline or ELPX+US. The longest tumor doubling time occurred in mice treated with DoxLPX+US (e.g., 9.2±2.7 days) which was significantly longer than the doubling time of tumors treated with free Dox alone, and longer than LDox treated group (p=0.06).

Kaplan-Meier survival curves were created for the six (6) experimental groups. There was rapid fall-off in survival in both saline and ELPX+US groups and by day 28, nearly 90% were dead. In contrast, all mice were still alive in the DoxLPX+US group at day 28. Overall median survival was significantly longer in the DoxLPX treated mice (38.5 days) compared to mice receiving free Dox (28 days), LDox (28 days), saline (24.5 days), or and ELPX+US (26.3 days) mice. The median survival time of LDox+MB+US group was numerically, but not significantly, longer compared to the saline, free Dox, or LDox groups. See, FIG. 4B.

D. Cardiac Function Assessments

Serial echocardiographic images were evaluated to determine cardiac function by measuring ejection fraction, fractional shortening and left ventricular mass of mice in all the six (6) experimental groups. For example, the measurements were compared between and within groups up to Day 21. Control mice treated with either saline or ELPX+US had stable left ventricular (LV) systolic function, fractional shortening, and LV mass. See, FIGS. 5A-C.

Mice receiving free Dox had a progressive decline in ejection fraction (EF) and fractional shortening (FS), such that by Day 21, these indices of systolic function were significantly less as compared to baseline. Similarly, LV mass tended to increase in the free Dox group, and was significantly greater than baseline by Day 14. In mice receiving co-injection of LDox and MB+US, there was a trend towards a decrease in ejection fraction and fractional shortening, and an increase in LV mass by Day 14. Mice receiving LDox did not have a change in systolic function but had an increase in LV mass, which was significant at Day 14 compared to baseline. Notably, mice treated with DoxLPX+US had stable ejection fraction, fractional shortening, and LV mass. These findings suggest that DoxLPS+US results in the least reduction in cardiac function of all the other Dox treatments.

The incidence of significant decreases in EF and FS, and increases in LV mass as a function of treatment group was plotted. When comparing the four (4) Dox groups, the DoxLPX+US group fail to show any abnormal changes in these metrics. In contrast, the free Dox, LDox and LDox+MB co-injection+US groups were all observed to develop abnormalities in these metrics. In particular, the free Dox group had the most mice developing some form of cardiac abnormality (e.g., p<0.05 vs. DoxLPX+US for abnormal decrease in FS).

The data suggest that a limited fraction of free Dox release (15˜25%) from DoxLPX after sonication indicates most drug content is still attached on the LPX debris. Similar to Doxil®, LPX encapsulates Dox into a micro-scale particle which reduces drug leakage through the capillary system of the heart tissue and diffusion into the cardiomyocytes thereby preventing free Dox-induced cardiac toxicity.

Although it is not necessary to understand the mechanism of an invention, it is believed that DoxLPX+US treatment may induce a stronger tumor inhibition than the equivalent dose of free drug because:
(1) DoxLPX achieves a local release of Dox in the tumor that can lead to more drug exposure to the tumor than normal organs;
(2) UTMC led to sonoporation that increases the drug permeation through the tumor vasculature and interstitium; and/or
(3) Micron-sized drug-containing DoxLPX debris retained in the malformed tortuous tumor vasculature after ultrasound-mediated MB destruction that achieved a locally high drug concentration in the tumor.
(4) Increased efficacy of UTMC and debris retention as a result of paracellular endothelial permeability.
(5) Ultrasound beam navigation concentration of a drug into the tumor resultant from a local drug release only occurs in the presence of ultrasound, as opposed to a diffuse systemic distribution of free Dox.
   Previously, it has been reported that US has very little effect on the drug release from LDox or LDox+MB without a chemical conjugation. Yu et al., “Low Intensity Ultrasound Mediated Liposomal Doxorubicin Delivery Using Polymer Microbubbles” Mol Pharmaceut. 2016; 13(1):55-64.

The data presented herein show that the free Dox group resulted in an impaired left ventricular function toward the decreases in average EF and FS. See, FIG. 5A, B. The variance of the cardiac function change between individual animals in the same group is notable. In humans, the criteria to define reduced cardiac function includes a decrease in EF by more than 10 percent to a final value of <50%. Rahman et al., “Anthracycline-induced cardiotoxicity and the cardiac-sparing effect of liposomal formulation” Int J Nanomed. 2007; 2(4):567-583.

Because the cardiac contractile function of mice is different, this criteria was slightly adjusted from that applied to moderate or severe human cardiac function changes.

Using echocardiography data with the control group serving as a baseline, 40% and 30% mice in Dox group showed a severe reduction in EF and FS on day 21, respectively. See, FIGS. 5D, E. In the LDox and LDox+MB+US groups, 12.5% and 22.2% mice showed a moderate decrease in EF, and both groups showed an 11.1% decrease in FS. I In the DoxLPX+US treatment group, no mice showed a substantial EF or FS change over 21 days as compared to the control group (saline) or ELPX+US group. The incidence rate showed an FS decrease in the Dox treatment group was significantly higher than that of DoxLPX+US group assessed by the Fisher's exact test (p<0.05).

Significant increases in LVIDs, LVIDd, and LV mass index in Dox treated mice were observed on Day 14 or Day 21 as compared to Day 0. See, FIG. 5C, 5F. Because LV mass is influenced by body size, LV mass index (indexation of LV mass to weight) provides more prognostic information. Dox and LDox treated mice showed significantly increased LV mass index on day 14. See, FIG. 5C. 20.0% and 11.1% of these groups showed a severely increased

LV mass index. 33.3% in LDox+MB+US group showed LV mass index showed a moderately increased LV mass index. See, FIG. 5F. DoxLPX+US, untreated control, and ELPX+US groups did not show any remarkable LV mass index change.

The administration of free Dox is usually associated with acute cardiac edema and LV dilation that may cause the LV mass and LV mass index increased in the early stage (5 weeks) post-administration, but both of these parameters eventually decrease in the late stages of treatment (20 weeks). Farhad et al., “Characterization of the Changes in Cardiac Structure and Function in Mice Treated With Anthracyclines Using Serial Cardiac Magnetic Resonance Imaging” Circ-Cardiovasc Imag. 2016; 9(12). The free Dox treated group showed disorganization of myofibrillar arrays and vacuolization in cardiac tissue. The replacement of chromatin by pale filaments known to be features of Dox-induced cardiomyopathy were also observed. These features impair the function of the myocardium and are not observed in the LDox, DoxLPX+US and LDox+MB+US groups.

E. Histological Analyses

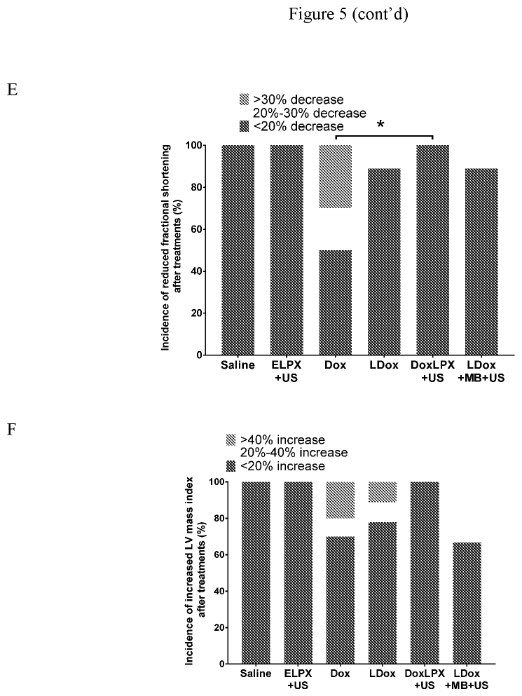

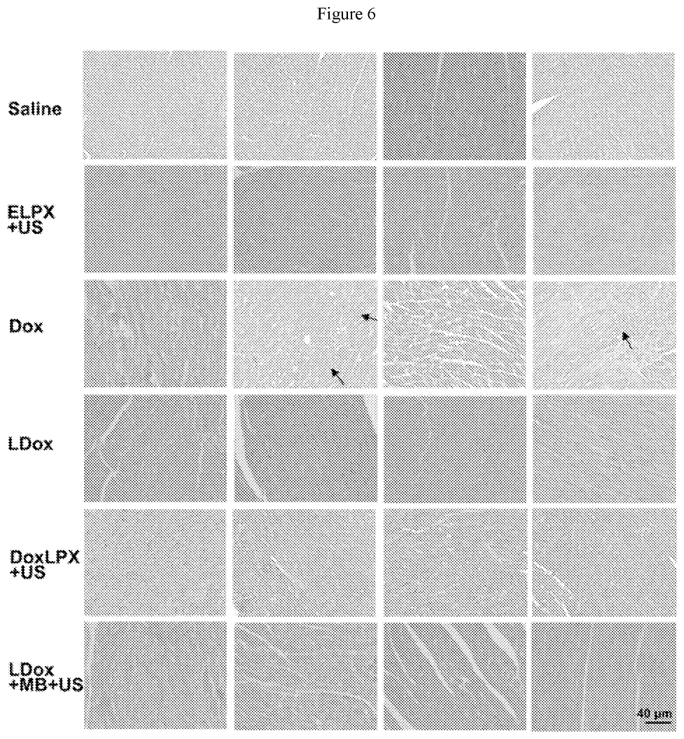

The DoxLPX+US, LDox, and LDox+MB+US treatments did not show noticeable histological damage to the heart tissue subsequent to hematoxylin/eosin staining. See, FIG. 6. In contrast, an equivalent dosage of free Dox led to disorganization of myofibrillar arrays (first left panel of Dox group) and focal cytoplasmic vacuolization (black arrows).

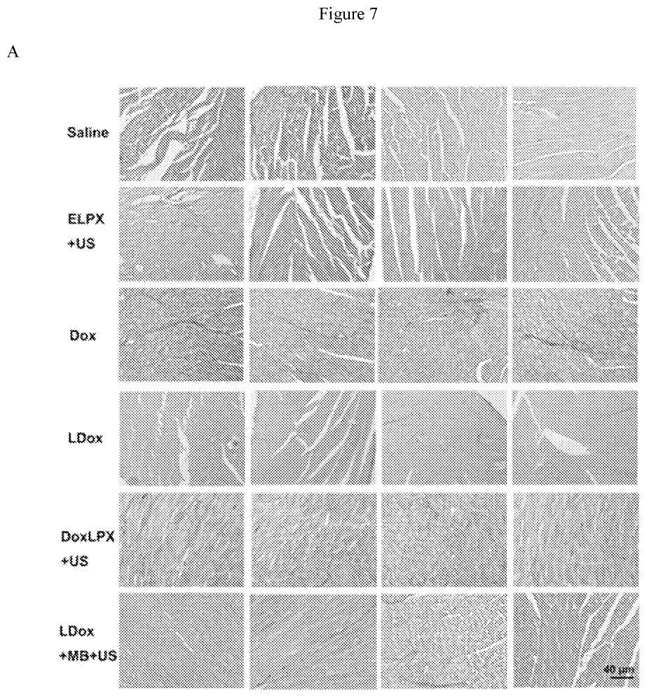

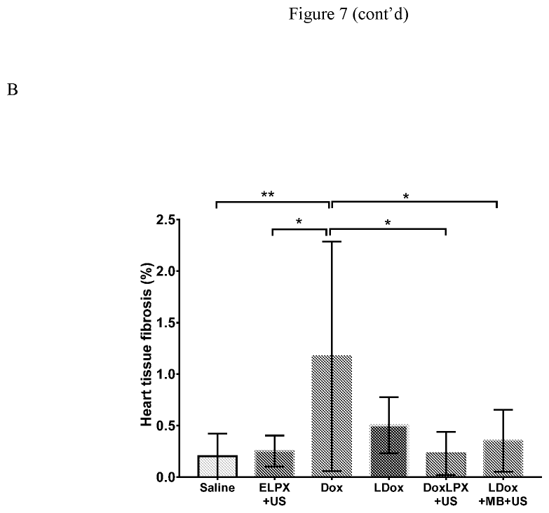

Furthermore, free Dox administration induced moderate myocardial interstitial and perivascular fibrosis. See, FIG. 7A. The mice treated with DoxLPX+US, ELPX+US or saline had significantly lower collagen deposition in the heart tissue compare to Dox treated mice. An equivalent dosage of LDox or LDox+MB+US attenuated the fibrosis numerically, but the difference was not statistically significant. See, FIG. 7B.

The toxicities of ELPX, LDox, DoxLPX and their combinations were examined with US sonication against MCA205 sarcoma cells in vitro. ELPX, LDox, DoxLPX or free Dox at 8 μg/mL did not change the cell membrane integrity. UTMC results in an instant cell viability change and induces lower cell survival/proliferation as compared to the control or the non-US treated counterparts. It has been suggested that UTMC could generate resealable pores on the cell membrane and many cells can still survive. Helfield et al., “Biophysical insight into mechanisms of sonoporation” P Natl Acad Sci USA. 2016; 113(36):9983-9988.

F. Caspase Activity

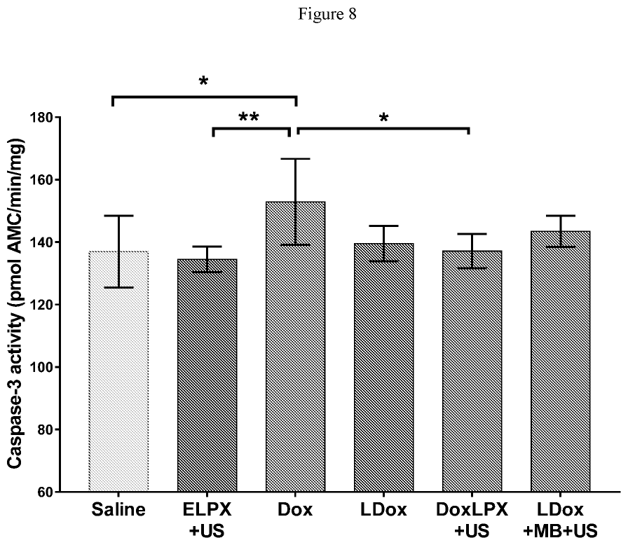

Caspase activity, a marker of apoptosis, was quantified by fluorescence intensity of the substrate peptide for caspase-3. Caspase activity in cardiac tissue of mice receiving free Dox was higher than that in mice treated with DoxLPX+US; indeed, myocardial caspase activity in DoxLPX+US treated mice was no different from that in control mice receiving ELPX+US or saline. See, FIG. 8. Cardiac caspase activity in mice receiving LDox or LDox+MB coinjection+ultrasound was numerically lower, but not statistically different, than that in mice receiving free Dox.

Fibroblasts are believed to infiltrate and proliferate in healed myocarditis. Dox treated mice showed significantly more areas of patchy myocardial interstitial fibrosis and perivascular fibrosis than that of DoxLPX+US and LDox+MB+US groups. LDox and LDox+MB+US group showed numerically more fibrosis area than DoxLPX+US group. See, FIG. 7B.

Although it is not necessary to understand the mechanism of an invention, it is believed that cardiomyocyte damage may be at least one underlying structural basis for the observed reduction in myocardial function. A central marker for the activation of the caspase cascade, caspase-3 activity was used to measure the apoptosis in the heart tissue. A significant higher caspase-3 activity was found in the heart tissue treated with free Dox compared to control, ELPX+US and DoxLPX+US groups. See, FIG. 8. The LDox and LDox+MB+US groups showed a numerically lower caspase-3 activity as compared to the Dox group which may result in reduced cardiac function after these treatments.

G. Biodistribution of Cy5.5LPX+US

A small molecule fluorophore, Cy5.5-NH2 was used as a substitute for Dox to investigate the drug biodistribution and LPX carriers because: (1) the fluorescence of Dox at ex/em 480/590 nm was too weak for ex vivo imaging; and (2) the autofluorescence of tissue sample could influence the test sensitivity. Cy5.5-NH2 has many physical/chemical similarities compared to the Dox, including moderate water solubility, the primary amine group with the acid dissociation constant (pKa) slightly higher than 7, and similar molecular weight (Cy5.5-NH2, 754 Da vs. Dox, 544 Da).

Doxorubicin biodistribution resulting from the varying treatment regimens was assessed using bioluminescence imaging of organs (n=3) harvested 3.5 hours after treatment. The fluorophore Cy5.5 was used as a surrogate for Dox in the free, liposomal, and the two LPX MB formulations (e.g., Cy5.5, LCy5.5, Cy5.5LPX+US and LCy5.5+MB+US, respectively). Only the mice who received free Cy5.5 had noticeable Cy5.5 fluorescence in the heart and skeletal muscle. There was visible tumor uptake of Cy5.5 after Cy5.5LPX+US that was not seen in the other treatment groups. See, FIG. 9A.

The distribution of FITC was used to identify liposome distribution in the tumor 45 min post-treatment. See, FIG. 9A. Stronger fluorescence (green) was found close to the blood vessel (red) in tumors treated with LCy5.5+MB+US compared to the simple LCy5.5 administration. The debris of the LPX after UTMC are observed (green fluorescent dots) in the tumor vasculature 45 min after treatment. The saline perfusion could not flush the debris out of the vasculature.

The data show that Cy5.5LPX+US treatment increased the fluorescence signal in the tumor and lowered the signal in lung, heart, and spleen as compared to other Dox treatments. The highest drug concentration in the tumor of Cy5.5LPX+US treated group may be attributed to, or at least partially attributed to, the retention of the LPX debris in the tumor vasculature or due to extrusion of the debris into the interstitial space, where it could either be phagocytosed by tumor cells or slowly release the dox. For example, the drug-containing LPX debris may slowly release the drug and exert the efficacy by diffusing and degrading in the tumor.

Both the nano-scale liposomal Dox or micro-scale DoxLPX carrier reduced the drug extravasation through the capillary of the myocardium that protected the cardiomyocytes from its toxicity. LCy5.5 and LCy5.5+MB+US showed numerically or significantly more accumulation in lung and spleen compare to Cy5.5LPX treatment, possibly because phagocytes in spleen and lung internalized the 300 nm size liposome. The phagocytes in lung and spleen have less efficiency to internalize the micron-size particles than nano-scale particles. Gratton et al., “The effect of particle design on cellular internalization pathways” P Natl Acad Sci USA. 2008; 105(33):11613-11618. The albumin coating on the PLA shell of LPX may also increase the circulation time due to the reduced opsonin binding, therefore, decreasing the accumulation in the spleen and heart. Furumoto et al., “Effect of coupling of albumin onto surface of PEG liposome on its in vivo disposition” Int J Pharmaceut. 2007; 329(1-2):110-116; and Yokoe et al., “Albumin-conjugated PEG liposome enhances tumor distribution of liposomal doxorubicin in rats” Int J Pharmaceut. 2008; 353(1-2):28-34, respectively.

In one embodiment, the present invention contemplates a composition comprising a liposome-microbubble complex, approximately 3.5 μm in diameter. This complex is an effective carrier of Dox for US-mediated tumor therapy. DoxLPX+US treatment showed the lowest unwanted accumulation in heart, lung, and spleen and fast clearance from the blood, whereas liposomal drug and MB+liposomal drug co-administration+US only reduced the drug concentration in the heart. The treatment of DoxLPX and MB+LDox with UTMC led to greater inhibition of tumor growth, compared to the equivalent dosage of free Dox and LDox in the sarcoma tumor-bearing mice. DoxLPX+US treated mice showed the least damage to the cardiomyocytes and the lowest incidence rate of impaired cardiac function. In conclusion, DoxLPX developed in this study is a promising efficient platform to increase the antitumor efficacy and spare the dose-limiting reduction in cardiac function of free Dox. US-mediated LDox+MB co-administration is also an alternative to improve the chemotherapy efficacy of

LDox.

### EXPERIMENTAL

### Example I

### Preparation of Dox-Loaded Liposomes

Biotinylated Dox-loaded liposomes (liposomal Dox, LDox) were prepared using a 128:62:5:5 molar ratio of L-α-phosphatidylcholine, hydrogenated (HSPC), cholesterol (Chol), 1,2-distearoyl-sn-glycero-3-phosphoethanolamine-N-[biotinyl (polyethylene glycol)-2000] (DSPE-PEG-Biotin) and 1,2-distearoyl-sn-glycero-3-phosphoethanolamine-N-[methoxy(polyethylene glycol)-2000] (ammonium salt) (DSPE-PEG) (>99% purity, Avanti Polar Lipids Inc., Alabster, Ala.). This formulation is similar to the Doxil® liposome, i.e. HSPC:Chol:DSPE-PEG in a molar ratio of 112:76:10.18.

All lipids were dissolved in dichloromethane (DCM) in a glass vial and then dried under a stream of argon gas. The lipid thin film was hydrated in a 300 mM ammonium sulfate solution at a 10 mg/mL lipid concentration. The lipid film was then gently sonicated briefly (Sonicator 75D, VWR, Radnor, Pa.) and underwent five alternate freeze/thaw cycles by alternating immersion in liquid nitrogen and a 65° C. water bath. The resulting liposomes were then extruded through polycarbonate sizing filters (400 nm pore size, Nuclepore Track-Etch membrane, Whatman plc, Buckinghamshire, UK). The liposomes were purified by passing through a Sephadex G-25 column (PD-10 column, GE Healthcare Bioscience, Pittsburgh, Pa.).

Dox loading was performed as follows: A Dox solution (10 mg/mL) was added to the liposomes in a 9:25 weight ratio (Dox:lipid). The suspension was magnetically stirred and incubated at 65° C. for 1 h. Surplus (free) Dox was then removed by passing the suspension through Sephadex G-50 (20 mL gravity-flow chromatography column (Bio-Rad Laboratories, Hercules, Calif.) and Sephadex G-75 (PD-10 column, GE Healthcare Bioscience). The mean hydrodynamic diameter of the final liposomes was determined by dynamic light scattering (Zetasizer Nano, Malvern, Zetasizer, Worcestershire, UK). The concentration of the incorporated Dox was measured by fluorometry at excitation/emission wavelengths of 485/595 nm (Beckman Coulter, DTX-880 Multimode detector, Brea, Calif.) in 0.3% (v/v) Triton X-100 Tris-HCl buffer solution (50 mM, pH 7.0) to lyse the liposomes.

For visualization of the accumulation and retention of the liposomes in the tumor vasculature in in-vivo studies, FITC-labeled liposomes was prepared using a similar lipid formulation with HSPC, cholesterol, FITC-PEG-cholesterol (MW 1K, Creative PEGWorks, NC), DSPE-PEG-Biotin, and DSPE-PEG mixed in 128:42:20:5:5 molar ratios and the above procedure (without the Dox loading steps).

### Example II

### Preparation of Drug-Loaded Liposomal Microbubbles

Liposomal Dox-loaded polymer MBs (DoxLPX) synthesized (˜6×10⁻⁷ μg/MB, FIG. 1A-C). In vitro studies determined optimal US regime for DoxLPX. Doxorubicin (>98% purity) was purchased from Cayan Chemical (Ann Arbor, Mich.).

Liposomal Dox-loaded polymer microbubble complexes (DoxLPX and FITC-lipoplexes (FITC-LPX) were synthesized by conjugating biotinylated polymer MBs to biotinylated LDox using biotin-avidin linking chemistry. Yu et al., “Low Intensity Ultrasound Mediated Liposomal Doxorubicin Delivery Using Polymer Microbubbles” Mol Pharmaceut. 2016; 13(1):55-64. The polymer MBs (3.3 μm average diameter) were composed of an outer layer of crosslinked human albumin, an inner shell of (poly-D,L-lactide, PLA) and a core of nitrogen gas. Briefly, PLA was dissolved into isopropyl acetate and mixed with cyclooctane. The solution was slowly added into the stirred medical grade human serum albumin (HSA) at 30° C. and emulsified by pumping through a stainless-steel filter (7-micron pore size) by a peristaltic pump for 15 min. Then the emulsion was added in 0.07% glutaraldehyde solution under vigorous stirring for 4 hours. HSA on the emulsion droplets were quickly crosslinked and the organic solvent were evaporated from the solution. The result polymer MBs were washed 3 time by centrifuging the solution at 2,000 rpm for 10 min and subnatant below the MB layer was discarded.

LDox was conjugated to MBs via biotin-avidin interaction. Briefly, 6×10⁸ MBs were incubated 250 μL avidin (1.25 mg) in PBS at room temperature for 2 h, washed in PBS 3 times and centrifuged. Purified biotinylated LDox, synthesized as above (1.5 mL) was mixed with the avidinated MBs at room temperature for 2 h, then centrifuged. Excess (e.g., unconjugated) liposomes in the subnatant were discarded, yielding a final product of MBs carrying Dox-loaded liposomes (i.e., Dox-lipoplexes and/or DoxLPC) floating in PBS. The DoxLPX were counted and sized by a Coulter counter (Beckman Coulter, Multisizer 3, Brea, Calif.), and the amount of Dox loading was assessed by fluorometry using 0.3% Triton X-100 in Tris-HCl buffer solution (50 mM, pH 7.0). Conjugation of the liposome to the MB surface was verified by fluorescence microscopy (IX81, with a 40× objective, Olympus, Center Valley, Pa.) (FIG. 2B). A negative control microbubble formulation comprised empty liposomes (no Dox) conjugated to polymer microbubbles (empty lipoplexes, ELPX). For the biodistribution studies described below, FITC-LPX was prepared by conjugating FITC-liposome on the polymer MBs and purified using the same procedure as described for unlabeled DoxLPX.

### Example III

### In Vitro Ultrasound-Induced Dox Release from DoxLPX

Measurement of Dox release in response to ultrasound and various acoustic parameters, an in vitro experiment setup employing a degassed deionized water tank maintained at 37° C. and equipped with a calibrated transducer was used a previously described. Yu et al., “Low Intensity Ultrasound Mediated Liposomal Doxorubicin Delivery Using Polymer Microbubbles” Mol Pharmaceut. 2016; 13(1):55-64.

Ultrasound treatment was provided by a 1 MHz flat single element transducer (A302S, 25.4 mm in diameter Olympus NDT, Waltham, Mass.) immersed in the tank and excited with an arbitrary function generator (AFG3252, Tektronix, Beaverton, Oreg., USA) and a gated radio frequency power amplifier (250A250AM8, Amplifier Research, Souderton, Pa., USA). The ultrasound field was calibrated with a 200-μm capsule hydrophone (HGL-0200, Onda Corp, Sunnyvale, Calif., USA). Three ultrasound regimes were tested. See. Table 1. DoxLPX concentration was adjusted to 4.7×10⁶/mL in PBS with a loaded Dox concentration of 100 μg/mL. For each experiment, a 500 μL volume of DoxLPX suspended in PBS was gently magnetically stirred in a polystyrene tube positioned at 40 mm from the transducer surface.

### Example IV

### Cell Viability Assessment

Myocardial fibrosis and caspase-3 (apoptosis marker) in DoxLPX+US treated animals compared with that from control hearts. Data presented as mean±SD.

MCA 205 murine sarcoma cells (generously provided by Dr. Walter J. Storkus at the University of Pittsburgh School of Medicine) were cultured in RPMI-1640 (Lonza, Biowhittaker, Basel, Switzerland), supplemented with 10% heat deactivated fetal bovine serum (FBS) and 1% penicillin-streptomycin at 37° C., and sub-cultured every 2 days and used before 8 passage.

MCA 205 cells were suspended (5×10⁴ cells in 500 μL media) in capped sterile polystyrene tubes. One of the following formulations was added to the tubes: DoxLPX, LDox, free Dox, ELPX, LDox+MB (e.g, unconjugated LDox and MB). The DoxLPX, ELPX, LDox+MB (MB:cell ratio was 136:1) formulations were treated with ultrasound for 60 s using the pulses described in Table 1. Cell viability was evaluated 30 min later using the trypan blue exclusion assay where controls comprised identical tubes not exposed to US.

Live cells and total cell number were counted on a hemocytometer using an inverted microscope (Motic AE21, Scientific Instrument Company, Campbell, Calif.). Acute cell viability was quantified as the number of the live cells (trypan blue negative) divided by the total cell numbers. Each treatment condition was repeated independently three times.

After US delivery to the ELPX+US, DoxLPX+US, and LDox+MB+US formulations, the 2.5×10⁴ cells MCA 205 cells were placed in each well of a 24-well plate (Falcon Multiwell, Becton Dickinson, San Jose, Calif.) and returned to the incubator for a long-term cell viability assessment. Control conditions included cells mixed with free Dox, LDox, DoxLPX, ELPX without US treatment and untreated cells. After 1 h incubation, the wells were replaced with 500 μL fresh media. At 24, 48, and 72 h, the cells were incubated in 500 μL media containing 50 μg/mL alamaBlue® for 1 h. Thereafter, 100 μL media in each well was sampled and assayed on a plate reader (Beckman Coulter, DTX-880 Multimode detector, Brea, Calif.) at a wavelength of λex=530 nm and λem=590 nm. The same volume of media containing alamaBlue® was added in empty wells in the same 24-well plate to be used as the baseline absorbance. The percentage of viability at 24, 48, and 72 h was calculated by normalizing to that of untreated cells. As Dox-induced cytotoxicity takes about 48 hours to manifest, acute cytotoxicity (trypan blue assay) was attributed to ultrasound-microbubble interaction. He et al., “Self-Assembled Cationic Biodegradable Nanoparticles from pH Responsive Amino-Acid-Based Poly(Ester Urea Urethane)s and Their Application As a Drug Delivery Vehicle” Biomacromolecules. 2016; 17(2):523-537.

### Example II

### In Vivo Tumor Treatment

This example provides in vivo studies were performed using the Pulse 3 US regime. See, Table 1. Four (4) Dox-containing formulations (5 mg/kg equivalent Dox dosage per treatment) were given intravenously: i) free Dox; ii) liposomal Dox (LDox); iii) DoxLPX; and iv) MB+Ldox co-injection. The control groups were MB-liposome complexes loaded with either empty liposomes (ELPX) or saline only. Treatments were infused over 15 min via internal jugular indwelling catheter on Days 0, 3, 7, 10.

Tumor volume and cardiac function serially monitored with high-resolution US. Data presented as mean±SD.

Murine sarcoma model. The animal protocols were approved by the University of Pittsburgh Institutional Animal Care and Use Committee and conform to the Policy on Human Care and Use of Laboratory Animals. Female mice were used due to their susceptibility to Dox cardiotoxicity. Lipshultz et al., “Assessment of dexrazoxane as a cardioprotectant in doxorubicin-treated children with high-risk acute lymphoblastic leukaemia: long-term follow-up of a prospective, randomised, multicentre trial” Lancet Oncol. 2010; 11(10):950-961: and Lipshultz et al., “Female Sex and Higher Drug Dose as Risk-Factors for Late Cardiotoxic Effects of Doxorubicin Therapy for Childhood-Cancer” New Engl J Med. 1995; 332(26):1738-1743. Eight-week-old female C57BL/6 mice were obtained from Jackson lab and housed in the animal facility of the Division of Laboratory Animal Resources at the University of Pittsburgh. At 9 weeks of age, mice were inoculated subcutaneously with 1.5×10⁶MCA205 cells in the lower flank near the base of the tail and tumors were allowed to grow prior to treatment.

Therapeutic ultrasound. Concurrent with intravenous DoxLPX, MB+LDox, or ELPX MB delivery to a tumor, a combined US Pulse 3 (e.g, 1 MHz, 10-cycle duration at 1,000 kPa followed by 490-cycle duration at 170 kPa) was repeated five hundred (500) times every 2.5 s. See, Table 1.

A single-element immersion transducer (A303S, 12.7 mm in diameter, Olympus NDT), driven by an arbitrary function generator (AFG3252, Tektronix) connected to a gated radio frequency power amplifier (Model 250AM8, Amplifier Research) was used to deliver the therapeutic US. The US field was calibrated with a 200-μm capsule hydrophone (HGL-0200, Onda Corp).

Based on the in vitro drug release studies described above, Pulse 3 (see Table 1) was used for all in vivo studies. The pulse was repeated 500 times, with the pulse train repeated every 2.5 s to allow MB reperfusion into the target area. The overall duty cycle was 10%. The spatial peak temporal average intensity was 0.16 W/cm². The US treatment was continued for approximately 3-5 minutes after the intravenous MB infusion was completed to achieve complete MB destruction (e.g., ˜12-15 min total sonication time).

### Example III

### Assessment of Tumor Growth

Tumor growth was evaluated by high-resolution 3D US imaging of the tumor using automated scanning (Vevo2100, Visualsonics, Ontario, Canada) at 21 MHz with a 0.2-mm step size between cross-sections. Tumor cross-sections were manually outlined and tumor volumes were calculated from 3D reconstructions of the outlined area. This technique can measure subcutaneous tumors with an irregular shape with an accuracy of ±20 mm³. Tumor growth over time was fit to an exponential curve (y)=X₀·e^{kt}, where X₀ represents initial tumor volume, and t stands for time in days. Doubling times (DT) were calculated as DT=(Ln2)/k.

### Example IV

### Assessment of Cardiac Function

To evaluate relative effects of differing Dox treatments on cardiac function, serial echocardiography was performed.

Mice were lightly anesthetized with 0.9˜1.2% isofluorane until the heart rate stabilized at 400 to 500 beats per minute. Two-dimensional short-axis images were obtained using a high-resolution Micro-Ultrasound system (Vevo 2100, VisualSonics Inc.) in B-mode and M-mode from parasternal short-axis view. Left ventricular ejection fraction (LVEF), fractional shortening (FS), left ventricle (LV) end-diastolic volume (LVIDd), LV end-systolic volume (LVIDs), LV posterior wall diastolic thickness (PWd), LV posterior wall systolic thickness (PWs), and LV mass were calculated using the Vevo LAB software (version 1.7.1) in M-mode. LV mass index was calculated as LV mass/mouse body weight (g) on the day of measurement.

### Example V

### In Vivo Treatment of Tumor-Bearing Mice

Treatments with various Dox formulations commenced when tumor volume reached 40-90 mm³. The mice were randomly assigned to 1 of 6 treatment groups. Dox-containing formulations equivalently containing 278 μg/mL of: i) Dox (free Dox); ii) LDox; iii) DoxLPX; iv) co-injection of LDox+MB (1.7×10⁸ MBs) for a total of 100 μg per mouse; v) ELPX (empty liposomes conjugated to MBs); and vi) saline.

On the first day of treatment, the tumor bearing mice were anesthetized with 2% isoflurane in oxygen and an indwelling catheter was placed in the internal jugular vein to facilitate multiple intravenous infusions over time. See, FIG. 1B. The therapies (total volume 380 μL) were infused at 2.5 mL/h through the catheterized jugular vein, with the DoxLPX, ELPX, and LDox+MB co-injection formulations receiving concurrent US treatment on Days 0, 3, 7, and 10. See, FIG. 1B. Rarely, the catheter became occluded by the third or fourth treatment, requiring tail vein injection as an alternative route for drug infusion.

Treatment was given twice a week for two weeks (total 4 treatments; 20 mg/kg accumulated Dox dosage, excluding the saline and ELPX+US controls). Tumor volume and cardiac function were serially evaluated for up to 42 days using ultrasound imaging. Echocardiography was performed every 7 days after the 1st treatment. On day 42, or if tumor volume exceeded 1,600 mm³ before day 42, the mice were euthanized under deep anesthesia (5% isofluorane). Tumor and heart were harvested, washed with cold saline, then were cryopreserved or fixed with 10% formalin overnight, embedding in paraffin block and sectioning for further histology analysis.

### Example VI

### Biodistribution Assessment

To assess bio-distribution of the drug post ultrasound treatment, Cy5.5 was used as a fluorescent Dox analog, injected i.v. as: Cy5.5-NH₂, liposomal Cy5.5, liposomal Cy5.5+MB, Cy5.5LPX, and mice were euthanized 3.5 hrs later. Blood, tumor, spleen, kidney, liver, heart, lung, brain, skin on the back, muscle, femur (bone), and foot were imaged post-mortem and measured using a Xenogen IVIS 200 imaging system.

This example determines the fate of liposomal lipids and Dox after in vivo delivery. Cyanine5.5-amine (Cy5.5-NH₂, Lumiprobe, Hunt Valley, Md.) was used as a surrogate for Dox based on their molecular structure similarity, moderate water solubility, and strong near-infrared wavelength fluorescence. Liposomal Cy5.5-amine (LCy5.5) and Cy5.5-LPX were formulated using the same method used for LDox and DoxLPX preparation, respectively.

Separate C57BL/6J mice bearing 100-150 mm³ MCA 205 sarcoma tumors were intravenously injected with 360 μL of free Cy5.5-NH2, LCy5.5, Cy5.5LPX or LCy5.5+MB (co-injection) (equivalent 40 μg Cy5.5-NH2 per mouse) and treated with the same ultrasound regime as for the tumor suppression studies. At 3.5 h after treatment, 350-500 μL blood was collected from the orbital sinus, the mice were euthanized under deep anesthesia, and the organs were harvested. Specimens of blood, tumor, spleen, kidney, liver, heart, lung, brain, skin on the back, muscle, femur (bone), and foot were imaged using a Xenogen IVIS 200 imaging system. Kumar et al., “Pharmacokinetics and biodistribution of polymeric micelles containing miRNA and small-molecule drug in orthotopic pancreatic tumor-bearing mice” Theranostics. 2018; 8(15):4033-4049.

To further quantify the distribution of Cy5.5-NH₂, tumor, spleen, kidney, liver, heart, and lung were wet-weighed and 50 μg of each tissue specimen were homogenized in 200 μL lysis buffer (50 mM HEPES, 50 mM NaCl, 1% Triton X-100, 5 mM EDTA, and 15 mM DTT) using a sonicator homogenizer (Sonicator Ultrasonic Processor XL, Misonix, Farmingdale, N.Y.) in Eppendorf tubes immersed in an ice-water. The tissue samples were centrifuged at 12,000 rcf for 30 min and the blood samples were centrifuged at 1,500 rcf for 12 min at 4° C.

50 μL supernatant of each specimen was added in black 384 well plates. Cy5.5-NH2 fluorescence intensity of 3 parallel samples of each organ was determined at Ex/Em 680/710 nm using a plate reader (Infinite 200 PRO, Tecan, Männedorf, Switzerland). Free Cy5.5-NH₂ in the lysis buffer in the range of 0˜2 μg/mL was used to establish a standard curve.

To visualize the distribution of the liposomal drug carrier after US in the tumor, another group of tumor bearing mice was treated with the above Dox formulations where the liposomes were labeled with FITC: FITC-Liposome, FITC-LPX+US, and FITC-liposome+MB (co-injection)+US. Forty-five minutes after treatment, mice were intravenously administered 200 μL 2.5 mg/mL rhodamine-Lens Culinaris Agglutinin (Vector laboratories, Burlingame, Calif.), and15 minutes later, they were perfused with 4 ml saline through the aorta, then euthanized. Tumors were harvested and embedded in Tissue-Plus O.C.T. (Fisher HealthCare, Houston, Tex.) for microscopic analyses. 150-μm thick sections were prepared for imaging using a 20× oil-immersed objective mounted on the Nikon A1 confocal laser microscope system.

### Example VII

### Tumor and Cardiac Histopathology and Fibrosis Analysis

Routine hematoxylin and eosin (H&E) stained heart tissue slides were made for histologic examination. Sirius red/fast green staining was performed on heart tissue sections post-fixed in paraffin blocks. Twenty-three (23) Images were obtained on an Olympus IX81 microscope interfaced with a digital CCD camera (Olympus DP71) and quantitated using ImageJ 1.5 software.

### Example VIII

### Caspase-3 Activity Assay

Heart tissue was washed twice with cold PBS and lysed in lysis buffer (50 mM HEPES, pH 7.4, with 5 Mm CHAPS, and 5 mM DTT). Tissue lysates were centrifuged at 10,000 rcf for 15 min, and the total protein concentration was measured using a BCA assay kit (Thermo Fisher Scientific, MA). Caspase-3 activity assay was performed in triplicate in 96-well plates. For each protease assay, 200 μL assay buffer (100 mM HEPES, pH 7.2, 10% sucrose, 0.1% CHAPS, 1 Mm Na-EDTA, and 2 mM DTT) containing 50 μM Ac-DEVD-AMC (Enzo life, Farmingdale, N.Y.) was incubated with 50 μg lysate protein at 37° C. for 1 h.

Caspase-3 activity was measured by AMC liberation from Ac-DEVD-AMC substrate at 380/460 nm using a plate reader (Beckman Coulter, DTX-880 Multimode detector, Brea, Calif.). Relative fluorescence of substrate control was subtracted as background emission. Free AMC (Enzo life, NY) was used to make the standard curve:

$$Caspase - 3 activity ( n mol AMC / min / mL ) = \frac{n mol AMC}{t \times v} \\ v = sample volume of 0.2 mL . t = reaction time of 60 min .$$

### Example IX

### Statistical Methods

All data are expressed as mean±SD (e.g., standard deviation). Tumor volume and DT were compared using analysis of variance (ANOVA) on ranks because the growth rate of treated tumors was not normally distributed. Post hoc testing was performed using the Tukey method (Prism 7.0, GraphPad).

Ejection fraction (FS) and left ventricular (LV) mass index data within each treatment group were compared to that on day 0 (baseline) within the group using a two-tail t-test. The percentage of mice with changes in ejection fraction, fractional shortening and LV mass index, categorized as mild, moderate or severe, was compared between groups using Chi-square test, and differences between two groups were compared using Fisher's exact test. Kaplan-Meier survival curves were compared between groups using the log-rank test for trend.

We claim:

1. A method, comprising:
a) providing;
i) a patient comprising a cancerous tumor and at least one cardiac function parameter, said cancerous tumor having a volume;
ii) a pharmaceutically acceptable composition comprising a doxorubicin-lipopolycomplex and a polymer microbubble;
iii) an ultrasonic transducer configured to generate a plurality of combined ultrasonic waves, wherein said plurality of combined ultrasonic wave comprises a first ultrasonic pulse having a higher acoustic pressure than a second ultrasonic pulse;
b) injecting said pharmaceutically acceptable composition into said patient;
c) exposing said cancerous tumor to at least one of said plurality of combined ultrasonic waves concurrently with said injecting;
d) reducing said cancerous tumor volume without a clinical impairment of said at least one cardiac function parameter.

2. The method of claim 1, wherein said doxorubicin-lipopolycomplex is conjugated to said polymer microbubble.

3. The method of claim 1, wherein said at least one cardiac function parameter is selected from the group consisting of ejection fraction, fractional shortening, left ventricular systolic function and left ventricular mass.

4. The method of claim 1, wherein said clinical impairment of said at least one cardiac function parameter is greater than 30% of a baseline value.

5. The method of claim 1, wherein said polymer microbubble comprises a polylactide polymer and nitrogen gas.

6. The method of claim 1, wherein said polymer microbubble has an internal diameter ranging from 0.5 to 10 μm.

7. The method of claim 2, wherein said conjugation is via avidin-biotin.

8. The method claim 1, wherein said polymer microbubble is coated with an albumin.

9. The method of claim 1, wherein said first ultrasonic pulse has an acoustic pressure selected from the group consisting of at least 500 kPa, 500 to 1500 kPa, 900 to 1100 kPa and 1000 kPa.

10. The method of claim 1, wherein said second ultrasonic pulse has an acoustic pressure selected from the groups consisting of less than 250 kPa, 150-200 kPa, 200-400 kPa, 170-180 kPa and 174 kPa.

11. The method of claim 1, wherein said combined ultrasound wave has a frequency selected from the group consisting of 0.5 MHz to 1.0 MHz, 0.75 MHz to 1.25 MHz, 1.0 MHz to 10 MHz, 0.9 MHz to 1.1 MHz and 1.0 MHz.

12. The method of claim 1, wherein said combined ultrasonic wave has a cycle number selected from the group consisting of 5 to 20 cycles, at least 25 cycles, 25 to 1000 cycles, 1000 to 10000 cycles, 50 to 150 cycles and 95 to 105 cycles.
